(19) United States
(12) Patent Application Publication
(10) Pub. No.: US 2021/0256542 A1
(43) Pub. Date: Aug. 19, 2021

(54) METHODS OF PREDICTING EMOTIONAL RESPONSE TO SENSORY STIMULI BASED ON INDIVIDUAL TRAITS

(71) Applicant: KONIKU INC., San Rafael, CA (US)

(72) Inventors: F. Kennedy MCDANIEL, San Rafael, CA (US); Marius GUERARD, San Rafael, CA (US); Oshiorenoya E. AGABI, San Rafael, CA (US)

(21) Appl. No.: 17/271,566

(22) PCT Filed: Mar. 23, 2019
(86) PCT No.: PCT/US2019/023787

§ 371 Date: Feb. 25, 2021

Related U.S. Application Data
(60) Provisional application No. 62/647,395, filed on Mar. 23, 2018.
(60) Provisional application No. 62/655,682, filed on Apr. 10, 2018.

(51) Int. Cl. G06Q 30/02; A61B 5/00; A61B 5/16; A61B 5/0531; A61B 5/08; A61B 5/021; A61B 5/026; A61B 5/0205; G06K 9/00; G06Q 30/06; G16H 10/20; G16H 10/60; G16H 50/20; G16H 40/67; G16H 50/70; G06N 5/04

(52) U.S. Cl. CPC G06Q 30/0201; A61B 2503/12; A61B 5/163; A61B 5/0531; A61B 5/0816; A61B 5/021; A61B 5/026; A61B 5/4277; A61B 5/02055; A61B 5/165; G06K 9/00302; G06Q 30/0631; G16H 10/20; G16H 10/60; G16H 50/20; G16H 40/67; G16H 50/70; G06N 5/04; A61B 5/7267

(57) ABSTRACT

Disclosed here are methods and systems for assessing an emotional response of a subject or a group to a sensory stimulus. The methods employ models that infer emotional response based on individual traits of subjects or groups.

### CROSS-REFERENCE TO RELATED APPLICATIONS

This application claims the benefit, under 35 U.S.C. §§ 119(e)(1) and 119(e)(3), of U.S. provisional patent application Nos. 62/647,395 filed on Mar. 23, 2018 and 62/655,682 filed on Apr. 10, 2018; the entire disclosure (specification, drawings and claims) of which is hereby incorporated by reference in its entirety for all purposes.

### BACKGROUND

Evaluation of human emotions based their own expression have been a difficult task. Some emotions may be better defined by words, such as fear, or disgust, probably because they were taking an important part in the selective evolution process. For example, fear is what we feel when confronted to a danger so it may be an emotion shared by all independently of our culture. However, some are way more difficult to define, as happiness. It is particularly the case of emotions felt when smelling or tasting. Indeed, unlike for vision and color description, the vocabulary to describe these senses and their related emotions are vague or not well known. Therefore, it has been difficult so far to assess the emotions people are feeling when tasting or smelling based on their expression only with language. This application provides a technological improvement over existing ways of solving the problem.

Sensory stimuli, such as odor and taste, are known to affect different individuals in different ways, based on, for example, culture, ethnicity, gender and environment. It would be advantageous, in certain circumstances, to be able to predict how a particular individual will respond (e.g., either positively or negatively) to a given sensory stimulus. Such predictive power would be beneficial, for example, in inferring an individual's preference for products possessing sensory stimuli, and in marketing certain products to consumers.

### SUMMARY

In one aspect provided herein is a method for inferring an emotional response of a subject to a sensory stimulus comprising: a) for each of a set of subjects in a cohort of subjects: (i) exposing the subject to one or more sensory stimuli; (ii) eliciting and electronically recording subjective response data from the subject to each sensory stimulus and receiving the recorded subjective response data into computer memory; (iii) electronically measuring objective response data from the subject to each sensory stimulus and receiving the measured objective response data into computer memory, wherein subjective responses and objective responses indicate an emotional response to the sensory stimulus; (iv) receiving into computer memory responses including trait data about the subject; and (v) receiving into computer memory data about each sensory stimulus; wherein the received data for a subject constitutes a subject dataset; b) generating a training dataset by collecting the subject datasets; c) training a machine learning algorithm on the training dataset to produce a model that infers an emotional response of a subject based on one or more individual trait data; d) at a user interface associated with a target subject (e.g., who is not in the cohort), inferring an emotional response by the subject to a sensory stimulus based on individual trait data provided by the target subject.

In another aspect provided herein is a method comprising: a) determining a profile comprising trait information about a plurality of individual traits for each of one or more subjects or consumer groups; and b) for each of one or more sensory stimuli, wherein each stimulus is an odor or a taste, predicting emotional response by each of the subjects or consumer groups to each of the sensory stimuli, based on the trait information. In one embodiment the method further comprises: c) translating the predicted emotional responses into recommendations to each subject or consumer group about attractiveness of products incorporating the sensory stimuli.

In another aspect, provided herein is a method of generating an emotional response prediction model comprising: a) providing a dataset that comprises, for each of a plurality of subjects, data including: (i) a subject profile comprising data on a plurality of individual traits from the subject; (ii) sensory stimulus data for each of one or a plurality of sensory stimuli to which the subject is exposed; and (iii) emotional response data for each subject indicating emotional response by the subject to each of the sensory stimuli to which the subject is exposed, wherein the emotional response data comprises one or both of subjective response data and objective response data; and b) training a learning algorithm to generate a model that infers a subject's emotional response to a sensory stimulus based on the subject's profile. In one embodiment the sensory stimulus in an odor. In another embodiment the sensory stimulus is a taste. In another embodiment the emotional response comprises a subjective response comprising a linguistic expression selected from spoken, written, or signed. In another embodiment the emotional response data comprises one or a plurality of objective responses selected from the group comprising facial expressions, micro expressions, brain signals, electroencephalography (EEG) signals, functional magnetic resonance imaging (fMRI) signals, body chemical stimuli, body chemical production, pupil dilation, skin conductance, skin potential, skin resistance, skin temperature, respiratory frequency, blood pressure, blood flow, saliva production and flow rate, and any combination thereof. In another embodiment the emotional response comprises data derived from social media activity of the subject or a group to which the subject belongs. In another embodiment the emotional response is classified into a discrete or continuous range. In another embodiment the emotional response is classified as a number, a degree, a level, a range or a bucket. In another embodiment the emotional response is classified as an image selected by the subject from a group of images. In another embodiment the emotional response is classified as a subjective feeling verbalized by the subject. In another embodiment the emotional response is classified into a category within a set of discrete categories, wherein the discrete categories are hierarchically arranged from least positive to most positive emotional response. In another embodiment the set comprises any of 3, 4, 5, 6, 7, 8, 9 or 10 discrete categories. In another embodiment the set comprises two discrete categories, including a negative emotional response and a positive emotional response. In another embodiment the set comprises three discrete categories, including a negative emotional response, a neutral emotional response and a positive emotional response. In another embodiment the emotional response is classified into a multi-variate response, with each variable being measured on a range. In another embodiment the variables include one or a plurality of responses selected from love, submission, awe, disapproval, remorse, contempt, aggressiveness, and optimism. In another embodiment the emotional response is selected from one or more of: amused, blissful, calm, cheerful, content, dreamy, ecstatic, energetic, excited, flirty, giddy, good, happy, joyful, loving, mellow, optimistic, peaceful, silly, and sympathetic. In another embodiment the emotional response is selected from one or more of: angry, annoyed, apathetic, bad, cranky, depressed, envious, frustrated, gloomy, grumpy, guilty, irritated, melancholy, pessimistic, rejected, restless, sad, stressed, and weird. In another embodiment the individual traits include trait selected from genetic traits, epigenetic traits, proteomic traits, phenotypic traits, socio-economic traits, ethnic traits, sex/gender traits, self-identifying traits, geographical traits, environmental exposure traits, psychological traits, health status traits or personal traits. In another embodiment the subject profile is obtained by providing a questionnaire to the subject and receiving from the subject answers to questions on the questionnaire. In another embodiment the subject profile comprises DNA sequence information from the subject. In another embodiment the sensory stimulus data comprise data on at least any of 2, 5, 10, 50, 100, 200, 300, 400, or 500 different sensory stimuli. In another embodiment the sensory stimuli data indicates one or more olfactory receptors stimulated by the sensory stimuli. In another embodiment the sensory stimuli data indicates one or more olfactory receptors stimulated by a sensory stimulus and/or one or more olfactory receptors not stimulated by a sensory stimulus. In another embodiment the olfactory receptors are selected from one or more of the receptors listed in International Patent Publication WO 2018/081657. In another embodiment the sensory stimulus is a complex chemical stimulus that stimulates a plurality of different olfactory receptors. In another embodiment the sensory stimulus comprises volatile organic compounds. In another embodiment the sensory stimulus data indicates one or more taste receptors stimulated by the sensory stimulus. In another embodiment the sensory stimulus comprises a product, e.g., selected from a food or beverage, a consumer packaged good, a chemical, an agricultural product or an explosive. In another embodiment the number of subjects is at least any of 50, 100, 250, 500, 750 or 1000. In another embodiment the machine learning is unsupervised and emotional response is classified into one of a plurality of clusters based on both subjective responses and objective responses. In another embodiment the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In another embodiment providing the dataset comprises: (A) exposing each subject to a sensory stimulus (e.g., an olfactory stimulus or a gustatory stimulus); (B) measuring one or a plurality of subjective responses from the subject; and (C) measuring one or a plurality of objective responses from the subject. In another embodiment, the method comprises, before (A), making baseline measurements on subjective responses and objective responses of the subject. In another embodiment making baseline measurements comprises exposing the subject to one or more neutral stimuli. In another embodiment the neutral stimulus is the taste or odor of water. In another embodiment measuring the subjective response comprises asking the subject to describe his or her emotional state. In another embodiment measuring the subjective response comprises showing the subject a plurality of images and asking the subject to select an image that most closely corresponds to the subject's emotional state. In another embodiment measuring the subjective response comprises asking the subject to rank his or her emotional state on a numerical scale. In another embodiment measuring the objective response comprises measuring one or more of facial expressions, micro expressions, brain signals, electroencephalography (EEG) signals, electrocardiographic (EKG) signals, pulse rate, functional magnetic resonance imaging (fMRI) signals, body chemical stimuli, body chemical production pupil dilation, skin conductance, skin potential, skin resistance, skin temperature, respiratory frequency, blood pressure, blood flow, saliva flow rate. In another embodiment the subject is a human.

In another aspect, provided herein is a method of inferring an emotional response by a subject to each of one or more sensory stimuli, the method comprising: a) obtaining a subject profile comprising trait information about a plurality of individual traits of the subject; and b) executing a classification model as described herein on the profile to infer an emotional response by the subject to each of one or more sensory stimuli. In one embodiment the inferred emotional response includes a “positive” response, a “neutral” response or a “negative” response. In another embodiment the positive emotional response is selected from one or more of: amused, blissful, calm, cheerful, content, dreamy, ecstatic, energetic, excited, flirty, giddy, good, happy, joyful, loving, mellow, optimistic, peaceful, silly, and sympathetic. In another embodiment the negative emotional response is selected from one or more of: angry, annoyed, apathetic, bad, cranky, depressed, envious, frustrated, gloomy, grumpy, guilty, indifferent, irritated, melancholy, pessimistic, rejected, restless, sad, stressed, and weird. In another embodiment the neutral emotional response is indifference. In another embodiment the method further comprises: c) communicating to the subject a predicted emotional response to a product comprising the sensory stimulus, e.g., predicting a “negative” emotional response, a “neutral” emotional response or a “positive” emotional response.

In another aspect provided herein is a method comprising: a) selecting a subject or consumer group for whom: (i) one or a plurality of sensory stimuli is predicted to elicit a negative emotional response, wherein the prediction takes into account a profile comprising data about individual traits of the subject or consumer group; or (ii) one or a plurality of sensory stimuli is predicted to elicit a positive emotional response, wherein the prediction takes into account a profile comprising data about individual traits of the subject or consumer group; and b) for a product comprising the sensory stimulus, performing one or both of: (i) increasing the amount of one or a plurality of sensory stimuli predicted to elicit a positive emotional response; and (ii) decreasing the amount of one or a plurality of sensory stimuli predicted to elicit a negative emotional response. In one embodiment emotional response is measured in each of multiple dimensions, each dimension measured on a discrete or continuous scale, and wherein amounts of sensory stimuli in the product are altered to alter predicted emotional response on one or a plurality of different dimensions.

In another aspect, provided herein is a method comprising: a) in response to a query from a customer about a product line comprising products each of which comprises a different sensory stimulus, collecting from the customer a customer profile; b) executing a classification algorithm on the customer profile to predict which product in the product line is most likely to produce a desired emotional response by the customer; c) communicating to the customer a recommendation about the product most likely to produce the positive emotional response; d) receiving from the customer an order for the recommended product; and e) fulfilling the customer order. In one embodiment the query of step (a), the communicating of step (c) and the receiving of step (d) are conducted electronically.

In another aspect provided herein is method of inferring an emotional response by a consumer group to each of one or a plurality of sensory stimuli, the method comprising: a) obtaining a consumer group profile comprising trait information about one or a plurality of group traits of the consumer group; and b) executing a classification model as described herein on the profile to infer an emotional response by the consumer group to each of one or more sensory stimuli. In one embodiment the trait information comprises data on one or more traits selected from geographic area of residence, gender, age, ethnic background, income, occupation, education, household size, religion, generation, nationality and social class. In another embodiment wherein the inferred emotional response can be a “positive” response, a “neutral” response or a “negative” response. In another embodiment the neutral emotional response is indifference. In another embodiment the positive emotional response is selected from one or more of: amused, blissful, calm, cheerful, content, dreamy, ecstatic, energetic, excited, flirty, giddy, good, happy, joyful, loving, mellow, optimistic, peaceful, silly, and sympathetic. In another embodiment the negative emotional response is selected from one or more of: angry, annoyed, apathetic, bad, cranky, depressed, envious, frustrated, gloomy, grumpy, guilty, indifferent, irritated, melancholy, pessimistic, rejected, restless, sad, stressed, and weird.

In another aspect provided herein is method comprising: a) determining, for a consumer group, a consumer group profile comprising one or a plurality of consumer group traits; b) executing a classification algorithm on the consumer group profile to predict which sensory stimulus profile in a line of sensory stimuli profiles is most likely to elicit a positive emotional response from subjects in the consumer group; and c) fulfilling orders for products to be stocked at stores in a geographical area where the consumer group is likely to shop with products comprising a sensory stimulus profiles predicted to elicit the positive emotional response. In one embodiment the consumer groups are based on one or more of geographic area of residence, gender, age, ethnic background, income, occupation, education, household size, religion, generation, nationality and social class. In another embodiment the consumer group traits comprise genetic information. In another embodiment the genetic information comprises identification of allelic variants of one or more marker genes. In another embodiment the genetic information comprises single nucleotide polymorphisms. In another embodiment the consumer group traits comprise epigenetic information. In another embodiment the consumer group traits comprise phenotypic information. In another embodiment the consumer group traits comprise information related to environment or environmental exposure to a substance. In another embodiment the substance is selected from the group consisting of automobile exhaust, agricultural chemicals, pesticides and radiation.

In another aspect provided herein is method for providing a product to a customer, wherein a sensory stimulus is associated with the product, the method comprising: (a) obtaining from the customer a profile comprising data on a plurality of individual traits of the customer; (b) executing a classifier that infers emotional response to the sensory stimulus (e.g., a classifier produced by the method as described herein) on the profile to infer an emotional response by the subject to the sensory stimulus associated with the product; and (c) if the emotional response is positive, providing the product to the customer. In one embodiment, upon receipt of payment by the customer, providing the product to the customer, e.g., by shipping or by in-store pick-up.

In another aspect provided herein is product comprising an odor or taste profile customized for a subject or target market comprising chemical stimuli predicted to elicit a desired emotional response profile from the subject or target market.

In another aspect, provided herein is a method for updating an inference model to reflect changes in social preferences comprising: a) providing an initial dataset that comprises, for each of a set of subjects in a cohort: (i) data about at least one sensory stimulus; (ii) emotional response data from the subject to the sensory stimulus including: (1) subjective response data, and (2) objective response data; and (iii) subject trait data; b) scouring the web for data from media about emotional response to the sensory stimulus from one or more of the subjects and/or individuals sharing group traits with subjects and incorporating the scoured data into the training dataset as emotional response data for one or more of the subjects to produce a training dataset; c) training a machine learning algorithm on the training dataset to produce a model that infers an emotional response of a subject based subject trait data; d) iteratively updating the model by: (I) scouring the web for new data from media about emotional response to the sensory stimulus from one or more of the subjects and/or individuals sharing group traits with subjects; (II) removing existing social media data from the training dataset and incorporating the scoured new data to produce an updated training dataset; and (III) training the machine learning algorithm on the updated training dataset to produce an updated model that infers an emotional response of a subject based subject trait data. In one embodiment the model is iteratively updated at least any of once, twice, three times, four times, five times, six times, seven times, eight times, nine times or ten times over a period selected from one month, one year, eighteen months, two years, three years, five years or ten years.

In another aspect provided herein is a system comprising: (a) a computer comprising: (i) a processor; (ii) a memory, coupled to the processor, the memory storing a module comprising (1) a subject profile, including data about individual traits for the subject; and (2) a classification rule which, based on the subject profile, predicts an emotional response by the subject to a sensory stimulus; and (iii) computer executable instructions for implementing the classification rule on the profile; and, optionally, (b) a display.

In another aspect, provided herein is a computer readable medium in tangible, non-transitory form comprising machine-executable code that, upon execution by a computer processor, implements a classification rule generated by a method as described herein to predict emotional response to a sensory stimulus.

A method for providing a product to a customer, wherein a sensory stimulus is associated with the product, the method comprising: (a) obtaining from the customer a profile comprising data on a plurality of individual traits of the customer; (b) providing the data of step (a) to the system comprising a computer comprising a processor, memory, to the processor comprising a subject profile in a classifier as described herein and executable instructions for letting the classifier on the profile; (c) obtaining a prediction of the emotional response of the customer the product; and (d) if the emotional response is positive, providing the product to the customer. In one embodiment, payment is made, by the customer, upon provision of the product.

In another aspect, provided herein is a method for providing a product to a customer, wherein a sensory stimulus is associated with the product, the method comprising: (a) obtaining from the customer a profile comprising data on a plurality of individual traits of the customer; (b) providing the data of step (a) to a computer system as described herein; (c) obtaining a prediction of the emotional response of the customer the product; and (d) if the emotional response is positive, providing the product to the customer. In one embodiment, payment is made, by the customer, upon provision of the product.

Disclosed is a method for assessing a physiological state of a subject in response to a stimulus, comprising: a) analyzing a physiological signal from the subject in response to the stimulus; and b) analyzing a linguistic expression of the subject in response to the stimulus. In some cases, the physiological state comprises an emotional state of the subject. In some cases, the emotional state comprises happiness, surprise, anger, fear, sadness, or disgust. In some cases, the stimulus comprises touch, pain, vision, smell, taste, or sound, which is elicited by an object. In some cases, the stimulus comprises the smell or taste elicited by the object. In some cases, the object comprises a chemical compound. In some cases, the subject is a human.

In some cases, the method further comprises detecting the physiological signal from the subject using a sensor. In some cases, the physiological signal is selected from the group comprising facial expressions, micro expressions, brain signals, electroencephalography (EEG) signals, functional magnetic resonance imaging (fMRI) signals, body odors, pupil dilation, skin conductance, skin potential, skin resistance, skin temperature, respiratory frequency, blood pressure, blood flow, saliva, and any combination thereof. In some cases, the sensor is connected to the subject. In some cases, the sensor is an EEG electrode. In some cases, the method further comprises assigning the analyzed physiological signal to a corresponding physiological state. In some cases, the assigning uses a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the method further comprises analyzing a reference physiological signal from the subject in response to a reference stimulus. In some cases, the reference stimulus elicits a reference physiological state. In some cases, the method further comprises comparing the physiological signal from the subject with the reference physiological signal. In some cases, the method further comprises assigning the physiological signal from the subject to the reference physiological state, wherein the physiological signal from the subject is comparable to reference physiological signal. In some cases, the physiological signal from the subject is within ±50%, ±40%, ±30%, ±20%, ±10%, ±5%, ±2%, or ±1% of the reference physiological signal.

In some cases, the linguistic expression is spoken, written, or signed. In some cases, the linguistic expression is translated into text. In some cases, the subject is asked to state the subject's emotional state. In some cases, the subject is asked to assign the subject's emotional state to a numerical level. In some cases, the subject is asked to assign the subject's emotional state to one or more images associated with the emotional state. In some cases, the method further comprises assigning the analyzed linguistic expression to a corresponding physiological state. In some cases, the method further comprises analyzing a reference linguistic expression from the subject in response to a reference stimulus. In some cases, the reference stimulus elicits a reference physiological state. In some cases, the method further comprises comparing the linguistic expression from the subject with the reference linguistic expression. In some cases, the method further comprises assigning the linguistic expression from the subject to the reference physiological state, wherein the linguistic expression from the subject is comparable to reference linguistic expression. In some cases, the linguistic expression from the subject and the reference linguistic expression are assigned to the same value on a grading scale. In some cases, the linguistic expression from the subject is assigned to a value on a grading scale that is within ±50%, ±40%, ±30%, ±20%, ±10%, ±5%, ±2%, or ±1% of the value assigned to the reference linguistic expression on the same grading scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary method for assessing a physiological state of a subject in response to a stimulus.

FIG. 2 shows an exemplary emotional state flower of a human subject. Emotions fall on several different dimensions and are graded in intensity from most intense (inside) to least intense (outside).

FIG. 3 shows an exemplary mapping between a list of compounds and their corresponding emotions based on biological optimum and cultural influence.

FIGS. 4A-4C show an exemplary training data set useful in training a learning algorithm as disclosed herein.

FIG. 5 shows a computer control system that is programmed or otherwise configured to implement the methods provided herein.

FIG. 6 shows a schematic illustration of an artificial neural network (ANN).

FIG. 7 shows a schematic illustration of the functionality of a node within a layer of an artificial neural network or deep learning neural network.

FIG. 8 shows partition of black and white dots by a support vector machine.

### DETAILED DESCRIPTION

### I. Introduction

Provided herein are methods and products for predicting emotional responses of individuals and groups to sensory stimuli, such as odors and tastes.

Methods of predicting (also referred to herein as “inferring”) emotional response can involve training a machine learning algorithm on a dataset comprising individual profiles, sensory stimulus data and emotional response data to produce a classifier that infers emotional response to a chemical stimulus based on an individual or group profile. Such classifiers can be used to infer an emotional response. Inferences about emotional response can be used to predict responses of individuals and groups products, e.g., consumer products, and to customize products to produce chemical stimuli that are attractive to individuals and/or groups.

Disclosed here are methods and systems for assessing a physiological state of a subject in response to a stimulus. The physiological state can be an emotional state. The stimulus can be an external stimulus including touch, pain, vision, smell, taste, sound, and any combinations thereof, elicited by an object. For example, the stimulus can be the smell and/or taste elicited by an object (e.g., a chemical compound). The method can access an emotional state of a subject in response to a smell and/or taste stimulus. The emotional state can comprise happiness, surprise, anger, fear, sadness, or disgust. The emotional state can be further classified into one or more levels. For example, an emotional state (e.g., happiness) can be further classified into 10 numeric levels (e.g., 1 being the lowest happiness level and 10 being the highest happiness level). The subject can be a human subject.

To evaluate the physiological state (e.g., emotional state) of a subject in response to a stimulus (e.g., the smell and/or taste of an object), the stimulus can be mapped to the physiological state using the methods and systems disclosed herein. In some cases, other stimulus, such as music, images, or text can be used in the intermediate steps to train the algorithm.

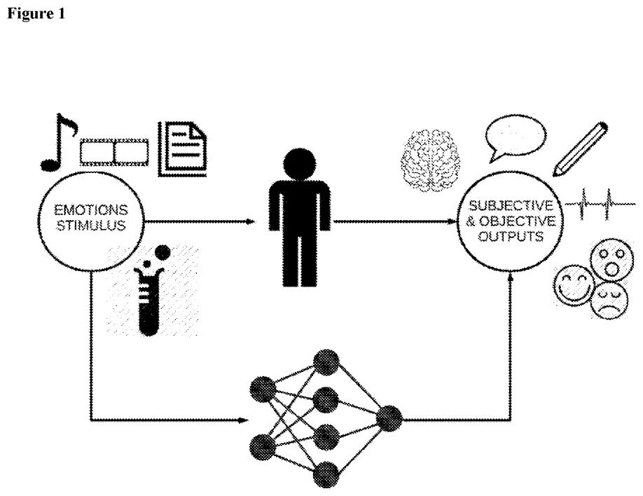

As shown in FIG. 1, the method can comprise an objective evaluation and/or a subjective evaluation. For example, the method can comprise analyzing a physiological signal from the subject in response to the stimulus. In another example, the method can comprise analyzing linguistic expressions of the subject in response to the stimulus. In yet another example, the method can comprise analyzing a physiological signal from the subject in response to the stimulus and analyzing linguistic expressions of the subject in response to the stimulus.

### II. Methods of Generating Classifiers to Infer Emotional Response to a Sensory Stimulus

Methods of generating classifiers to infer emotional responses to chemical stimuli can take advantage of machine learning. To construct these response prediction models (classification algorithms), a subject is exposed to a sensory stimulus (i.e., a taste or an odor) and the objective and/or subjective responses of the subject are assessed. A dataset is then created containing, for each of a plurality of subjects, (1) a subject profile comprising data on a plurality of traits (as described above) possessed by the subject, (2) a sensory stimulus to which the subject has been exposed (or data relating to said sensory stimulus); and (3) the emotional response(s) elicited in the subject by the sensory stimulus. The emotional response(s), along with the corresponding sensory stimulus or sensory stimulus data that evoked the responses, are entered into the database of the learning algorithm. In this way, a database is created which links particular individual traits with emotional responses elicited by a particular sensory stimulus.

In certain embodiments, classifiers (response prediction models) are created by obtaining subject information from a plurality of subjects, to provide a dataset. The number of subjects can be any of at least 2, 5, 10, 20, 30, 40, 50, 100, 200, 250, 500, 750, 1000, 5,000, 7,500, 10,000 or more (or any integral value therebetween) or more. The information can be obtained, e.g., orally or in written form, e.g., by providing a questionnaire to the subject and receiving from the subject answers to the questions on the questionnaire. Provision and completion of the questionnaire can be by hard copy or online.

A. Training Dataset

Methods of generating models to predict emotional response can involve providing a training dataset on which a machine learning algorithm can be trained to develop one or more models to predict emotional response. The training dataset will include a plurality of training examples, typically for each of a plurality of subjects and typically in the form of a vector. Each training example will include a plurality of features and, for each feature, data, e.g., in the form of numbers or descriptors. Where learning is to be supervised, the data will include a classification of the subject into a category of an emotional response to be inferred. For example, the emotional response may be “level of excitement” and the categories or classifications of this variable can be “excited” and “calm”. Typically, for machine learning, the training examples will have at least 10, at least 100, at least 500 or at least 1000 different features. The features selected are those on which prediction will be based.

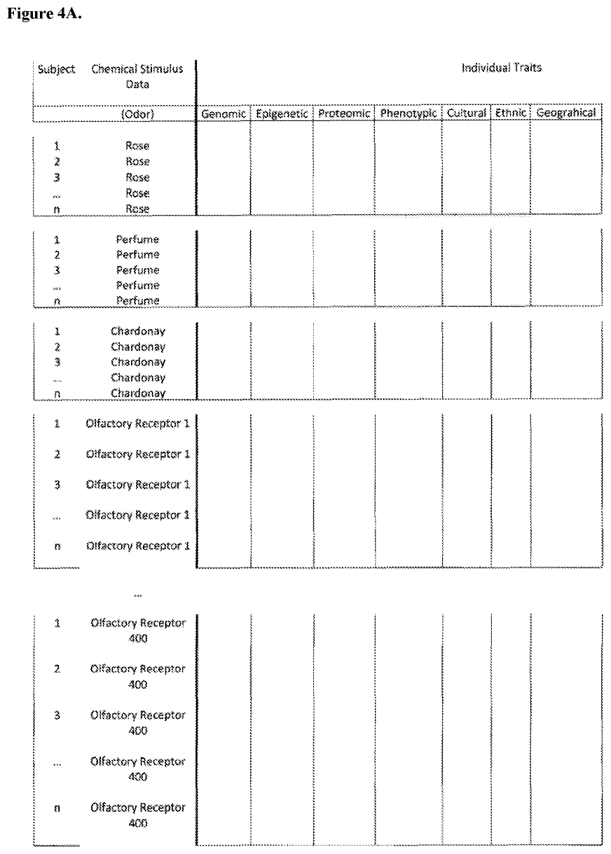

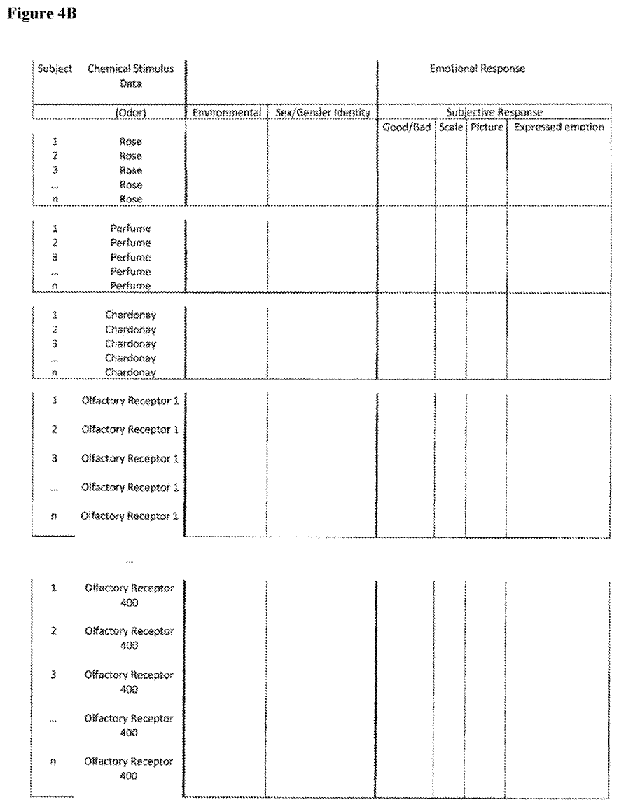

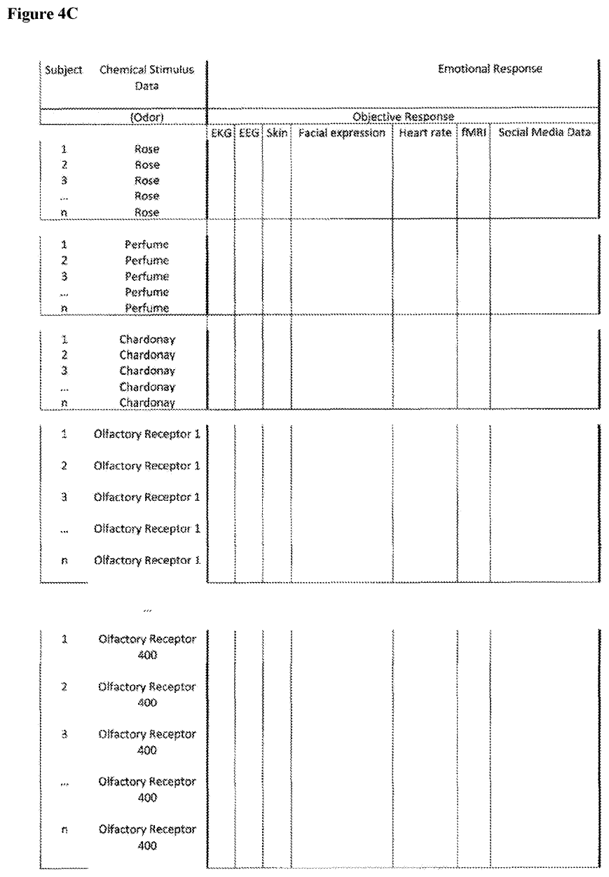

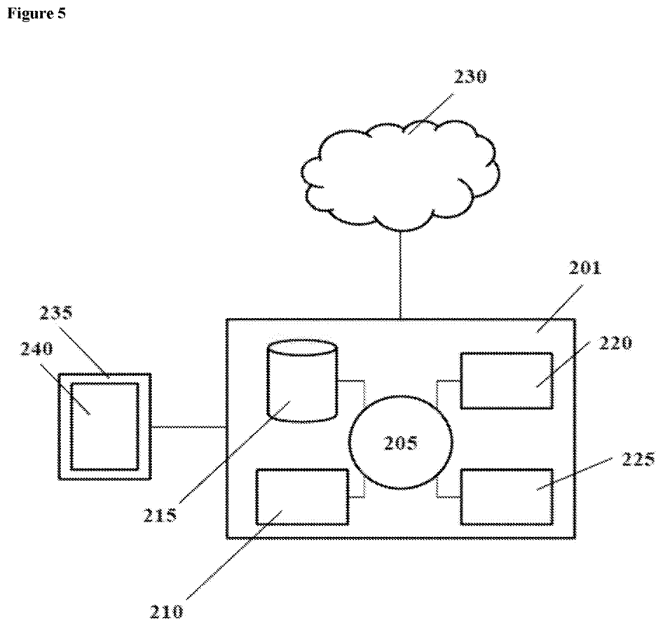

FIGS. 4A, 4B and 4C show an exemplary training dataset for use in training a learning algorithm to predict emotional response from subject or group profiles.

1. Subjects

A subject can be any organism that can respond to a stimulus, e.g., that possesses a sense of smell and/or a sense of taste. For example, the subject can be an animal such as a mammal, bird, reptile, amphibian or fish. Exemplary mammals include, for example, rodents, primates, carnivores, lagomorphs. Exemplary animals include, for example, dogs, cats, horses, bovines, pigs, sheep, and humans.

2. Subject Profile

Subject profiles are used in constructing the classifiers of the present disclosure by providing data including information on one or a plurality of individual traits. Any genomic, epigenetic, proteomic, phenotypic, ethnic, geographic, socioeconomic, sex/gender identity, or environmental information can be used as part of a subject profile. Exemplary subject traits are now described. The categories described herein are not meant to be mutually exclusive.

A subject profile (e.g., a list of traits possessed by a subject) is obtained by any method of communication (e.g., oral, written, signed) and is recorded, preferably digitally. For example, a subject is interviewed and the trait data provided by the subject is recorded (in writing, by audio or video recording, etc.) by the interviewer. Alternatively, a subject is provided with a questionnaire containing questions designed to elicit trait information, and the subject completes the questionnaire by providing answers to the questions in the questionnaire. A questionnaire can be, for example, a paper questionnaire (e.g., hard copy) or the questionnaire can be provided and completed online, or some combination of hard copy and online questionnaire provision and completion can be used.

a) Genomic Traits

Genomic traits include any information relating to the genome of the subject. Such information includes alleles of one or more particular genes, the sequence of one or more genes or chromosomes, the entire genome sequence of the subject; presence and number of tandem repeated sequences (e.g., trinucleotide repeats) and single nucleotide polymorphisms (SNPs).

In other embodiments, genomic traits can include partial genome sequences, e.g., sequences of exosomes, sequences of transcriptomes, sequences of cell free DNA.

SNP information can include, for example, SNPs known to be associated with certain traits such as diseases or anosmias. For example, genomic information can include information about genes known to be involved in the ability to smell certain compounds. Asparagus anosmia refers to the inability to smell asparagus in the urine. About 871 single nucleotide polymorphisms (SNPs) have been identified that are associated with this condition. These SNPs are located on chromosome 1—a chromosomal region that contains multiple genes connected to the sense of smell.

b) Epigenetic Traits

Epigenetic traits include modifications to cellular chromatin (i.e., genomic DNA packaged in histone proteins and non-histone chromosomal proteins). Such modifications include DNA methylation (e.g., cytosine methylation) and modification to histone and non-histone proteins including methylation, phosphorylation, ubiquitination, and glycosylation. Epigenetic information can methylation patterns of specified genes.

c) Proteomic Traits

Proteomic analysis provides information on the identity and quantity of proteins present in a particular cell, tissue or organ. It can also provide information about the post-translational modification of proteins present in a particular cell, tissue or organ. It also can include protein sequence variants.

d) Phenotypic Traits

Phenotypic information includes the physical characteristics of a subject, including, without limitation, age, gender, eye color, hair color, height, weight, body mass index, blood pressure, percent body fat, hormone levels (e.g., thyroid hormone, estrogen, estradiol, progesterone, testosterone), cholesterol level (e.g., total cholesterol, LDL, HDL, triglycerides), levels of circulating metabolites, levels of circulating ions (e.g., sodium, potassium, chloride, calcium), glucose levels (fasting and/or non-fasting), blood count, hematocrit, white cell count and vitamin levels. Additional phenotypic traits are known to those of skill in the art.

e) Ethnic Traits

Ethnic information includes information regarding the ethnicity of a subject. Ethnicity is state of belonging to a social group that has a common national or cultural tradition. More specific ethnic information relates to a tribe or band of which the subject is a member. Although certain types of ethnic information can overlap with certain types of geographical information, ethnicity is not always synonymous with geography, due to, for example, travel and migration. Ethnic traits can also include, for example, food and music preferences. Common ethnic groups in the United States include, for example, those listed in Table 1.

TABLE 1

| | | | |
|---|---|---|---|
| German | Hungarian | Salvadoran | Cambodian |
| Irish | Chinese | European | Syrian |
| English | Filipino | Jamaican | Arab |
| African American | Czech | Lebanese | Slovene |
| Italian | Portuguese | Belgian | Serbian |
| American | British | Romanian | Honduran |
| Mexican | Hispanic | Spaniard | Thai |
| French | Greek | Colombian | Asian |
| Polish | Swiss | Czechoslovakian | Pakistani |
| American | Japanese | Armenian | Nigerian |
| Indian | | | |
| Dutch | Austrian | Pennsylvania | Panamanian |
| | | German | |
| Scotch-Irish | Cuban | Haitian | Hmong |
| Scottish | Korean | Yugoslavian | Turkish |
| Swedish | Lithuanian | Hawaiian | Israeli |
| Other ancestries | Ukrainian | African * | Guyanese |
| Norwegian | Scandinavian | Guatemalan | Egyptian |
| Russian | Finnish | Iranian | Slavic |
| French Canadian | United States | Ecuadorian | Trinidadian and |
| | | | Tobagonian |
| Welsh | Asian Indian | Taiwanese | Northern |
| | | | European * |
| Spanish | Canadian | Nicaraguan | Brazilian |
| Puerto Rican | Croatian | Peruvian | Albanian |
| Slovak | Vietnamese | West Indian | Latin American * |
| White | Dominican | Laotian | Western |
| Danish | | | European * |

f) Geographic Traits

Geographic information includes information regarding the place of residence of a subject. Such information can be provided at one or more levels including, for example, Continental, country, region, state, city, town, neighborhood or street. For example, Continental level geographic information includes, for example, North American, Central American, South American, African, European, Asian, Pacific Islander, or Australian. Geographic information can limit a person's residence to than a defined area, such as, an area up to any of one square mile, 4 mi.², 25 mi.², 100 mi.², 625 mi.² or 10,000 mi.². Geographic information also can include, for example, site of residence (e.g., urban, suburban, wildland-urban interface),

g) Socio-Economic Traits

Socioeconomic traits include, for example, educational level, income, employment type, family structure, household size, religion, social class (e.g., caste, poverty, wealth), education, age.

h) Sex/Gender Identity Traits

As used herein, a sex trait refers to biological sex which can be male, female and intersex. Gender identity refers to a personal sense of traits referring to masculinity, femininity, transgender, and agender. Sexual preference refers to, without limitation, heterosexual, homosexual, bisexual, and asexual.

i) Environmental Traits

Environmental information includes information regarding the physical properties and climate of the location at which a subject resides. Examples of environments include, e.g., coastal, forest, riparian, desert, jungle, etc. Climate types include, for example, temperate, tropical, arctic, desert and Mediterranean. Climatic properties include, temperature, humidity, annual rainfall, cloudiness, solar exposure, and wind speed. Environmental information also includes the expososome, that is, the universe of environmental elements to which was exposed. Such elements include, for example, agricultural and industrial substances, airborne pollutants (such as automobile exhaust) and waterborne pollutants (such as fertilizers, pesticides and other agricultural chemicals).

j) Psychological Traits

Psychological traits can include measures of personality traits, e.g., the so-called “big five” personality traits of openness, conscientiousness, extraversion, agreeableness and neuroticism. Psychological traits also can include measures of clinical diagnosis of mental disorders, for example, as defined in the Diagnostic and Statistical Manual of mental disorders. Psychological traits further can include human behaviors including addictions, e.g., to cigarettes or alcohol.

k) Health Traits

Health traits include measures of human health including biometric data, pathological conditions, e.g., cancer, diabetes, heart disease, dementia, chronic lower respiratory diseases, stroke and kidney disease.

l) Personal Traits

Personal traits can include personal preferences in any of a number of areas including, for example, preferences in food and beverages, music, entertainment.

3. Group Profile

In certain embodiments classifiers developed by the methods herein are used to infer emotional responses of persons belonging to certain groups. Such groups can be defined as persons sharing any of the individual traits as discussed herein. For example, a group can be defined by ethnicity, or geographical location. Typically, other traits will be disproportionately represented in groups compared to the population as a whole. In inferring an emotional response of a group to sensory stimulus, a classifier can operate on a group profile analogous to an individual profile. The group profile will include as features traits that are shared or predominant in members of the group. For example, if ethnicity is an individual trait used in developing a classifier, then, ethnicity can be used as a feature in a group profile on which an inference of emotional response will be inferred. To the extent different traits cluster within groups, these traits also can be included in the group profile. Press control S go to sleep

4. Sensory Stimulus Data

a) Sensory Stimuli

The term “sensory stimulus,” as used herein, refers to any stimulus of the five senses, in particular, chemical stimuli of the sense of smell and the sense of taste. Such chemical stimuli can be referred to as odors or tastes. Odors also can be referred to as olfactory stimuli. Tastes can be referred to as gustatory stimuli. Odors are detected and transduced by olfactory receptors, some of which are located in the nasal passages. Exemplary olfactory receptors are listed in International Publication WO 2018/081657. Tastes are detected and transduced by gustatory receptors (located on taste buds) located on the lingual (tongue), buccal (inner cheek) and palatal (roof of mouth) surfaces of the oral cavity. Olfactory receptors can also contribute to taste.

Part of the data used to create the algorithms disclosed herein comprises data relating to sensory stimuli. Sensory stimuli can include odors and tastes. Accordingly, sensory stimulus data includes identification of chemical compounds (and combinations thereof) that produce particular odors. Sensory stimulus data also includes identification of one or a plurality of olfactory receptors that are stimulated by a particular chemical compound or combination of compounds. Alternatively, or in addition, sensory stimulus data includes identification of one or a plurality of olfactory receptors that are not stimulated by a particular chemical compound or combination of compounds. Exemplary compounds that stimulate olfactory receptors are volatile organic compounds (VOCs).

Sensory stimuli data can include specific information about the particular composition this can include, for example, the identity of chemicals in the composition as well as their chemical characteristics such as class of chemical compounds to which they belong in the relative amounts of each chemical in the composition constituting the sensory stimulus.

A sensory stimulus such as an odor or taste can be simple or complex. For example, an individual compound can serve as a sensory stimulus. Alternatively, odors comprising many different chemical compounds can serve as a sensory stimulus. For example, a tea-soaked cake comprises a complex mixture of compounds that can elicit a complex emotional response. Perfume, wine, and various scented consumer products also can include complex mixtures of smells and can serve as a sensory stimulus.

In additional embodiments, a physiological response to an odor is determined by identifying one or more olfactory receptors stimulated by the odor. See, for example, co-owned U.S. provisional patent application No. 62/655,682 filed Apr. 10, 2018.

Specific sensory stimuli include, for example, those listed in the following Tables 2a-2c.

TABLE 2a
Odorant compounds produced by fruit or plants.

| Compound Name | CAS # |
|---|---|
| alpha-ionone | 127-41-3 |
| alpha-phellandrene | 99-83-2 |
| alpha-pinene | 7785-70-8 |
| benzaldehyde | 100-52-7 |
| beta-ionone | 14901-07-6 |
| beta-pinene | 18172-67-3 |
| butyric acid | 107-92-6 |
| caryophyllen | 87-44-5 |
| damascenone | 23726-93-4 |
| delta-decalactone | 705-86-2 |
| e-2-hexenal | 6728-26-3 |
| ethyl butyrate | 105-54-4 |
| gamma-decalactone | 706-14-9 |
| geranial | 5392-40-5 |
| geraniol | 106-24-1 |
| hexanoic acid | 142-62-1 |
| hexyl acetate | 142-92-7 |
| limonene | 138-86-3 |
| linalool | 78-70-6 |
| mesifuran | 4077-47-8 |
| methyl anthranilate | 134-20-3 |
| methyl butyrate | 623-42-7 |
| neral | 5392-40-5 |
| nerolidol | 7212-44-4 |
| raspberry ketone | 5471-51-2 |

TABLE 2b
Odorant receptors for fruit-specific volatile compounds.

| | | | Literature | GenBank | Literature | |
|---|---|---|---|---|---|---|
| Odorant | CAS # | Organism | code | ID | Indication | Reference |
| limonene | 138-86-3 | Apolygus | AlucOR46 | NM_001190 | Tuned to six plant | Zhang Z, Zhang M, Yan |
| | | lucorum | | 564.1 | volatiles: | S, Wang G, Liu Y. A |
| | | (Meyer-Dür) | | | (S)-(−)-Limonene, | Female-Biased Odorant |
| | | | | | (R)-(+)-Limonene, | Receptor from Apolygus |
| | | | | | (E)-2-Hexenal, | lucorum (Meyer-Dür) |
| | | | | | (E)-3-Hexenol, | Tuned to Some Plant |
| | | | | | 1-Heptanol, and | Odors. Int J Mol Sci. |
| | | | | | (1R)-(−)-Myrtenol | 2016 Jul. 28; 17(8). pii: |
| | | | | | | E1165. doi: |
| | | | | | | 10.3390/ijms17081165. |
| | | | | | | PubMed PMID: |
| | | | | | | 27483241; PubMed |
| | | | | | | Central PMCID: |
| | | | | | | PMC5000588. |
| limonene | 138-86-3 | Megoura | OBP3 from | KT750882.1 | (E)-β-farnesene | Northey T, Venthur H, |
| | | viciae and | M. viciae | | (−)-α-pinene, | De Biasio F, Chauviac |
| | | Nasonovia | | | β-pinene, and | F X, Cole A, Ribeiro K A |
| | | ribisnigri | | | limonene | Junior, Grossi G, |
| | | | | | | Falabella P, Field L M, |
| | | | | | | Keep N H, Zhou J J. |
| | | | | | | Crystal Structures and |
| | | | | | | Binding Dynamics of |
| | | | | | | Odorant-Binding Protein |
| | | | | | | 3 from two aphid species |
| | | | | | | Megoura viciae and |
| | | | | | | Nasonovia ribisnigri. Sci |
| | | | | | | Rep. 2016 Apr. |
| | | | | | | 22; 6: 24739. doi: |
| | | | | | | 10.1038/srep24739. |
| | | | | | | PubMed PMID: |
| | | | | | | 27102935; PubMed |
| | | | | | | Central PMCID: |
| | | | | | | PMC4840437. |
| limonene | 138-86-3 | Marucavitr | MvitGOBP1- | NP_0011401 | MvitGOBP1-2 had | Zhou J, Zhang N, Wang |
| | | ata Fabricius | 2 | 85.1 | different binding | P, Zhang S, Li D, Liu K, |
| | | (Lepidoptera: | | | affinities with 17 | Wang G, Wang X, Ai |
| | | Crambidae) | | | volatile odorant | H. Identification of Host- |
| | | | | | molecules | Plant Volatiles and |
| | | | | | including butanoic | Characterization of Two |
| | | | | | acid butyl ester, | Novel General Odorant- |
| | | | | | limonene, 4- | Binding Proteins from |
| | | | | | ethylpropiophenone, | the Legume Pod Borer, |
| | | | | | 1H indol-4-ol, | Maruca vitrata Fabricius |
| | | | | | butanoic acid octyl | (Lepidoptera: |
| | | | | | ester, and 2 | Crambidae). PLoS One. |
| | | | | | methyl-3- | 2015 Oct. 30; 10(10): |
| | | | | | phenylpropanal | e0141208. doi: |
| | | | | | | 10.1371/journal. |
| | | | | | | pone.0141208. |
| | | | | | | eCollection 2015. |
| | | | | | | PubMed PMID: |
| | | | | | | 26517714; PubMed |
| | | | | | | Central PMCID: |
| | | | | | | PMC4627759. |
| limonene | 138-86-3 | Vinegar fly | Odorant | NP_525013.2 | Single dedicated | Dweck H K, Ebrahim S A, |
| | | Drosophila | receptor | | olfactory pathway | Kromann S, Bown D, |
| | | melanogaster | Or19a | | determines | Hillbur Y, Sachse S, |
| | | | | | oviposition fruit | Hansson B S, Stensmyr |
| | | | | | substrate choic | M C. Olfactory |
| | | | | | | preference for egg laying |
| | | | | | | on citrus substrates |
| | | | | | | in Drosophila. Curr Biol. |
| | | | | | | 2013 Dec. 16; 23(24): |
| | | | | | | 2472-80. doi: |
| | | | | | | 10.1016/j.cub.2013. |
| | | | | | | 10.047. Epub 2013 Dec |
| | | | | | | 5. PubMed PMID: |
| | | | | | | 24316206. |
| linalool | 78-70-6 | Bombyx | BmorOR-19 | NP_0010917 | Tuned to the | Groβe-Wilde E, Stieber |
| | | mori | | 85.1 | detection of the | R, Forstner M, Krieger J, |
| | | | | | plant odor linalool | Wicher D, Hansson B S. |
| | | | | | | Sex-specific odorant |
| | | | | | | receptors of the tobacco |
| | | | | | | hornworm manduca |
| | | | | | | sexta. Front Cell |
| | | | | | | Neurosci. 2010 Aug 3; 4. |
| | | | | | | pii: 22. doi: |
| | | | | | | 10.3389/fnce1.2010.0002 |
| | | | | | | 2. eCollection 2010. |
| | | | | | | PubMed PMID: |
| | | | | | | 20725598; PubMed |
| | | | | | | Central PMCID: |
| | | | | | | PMC2922936. |

TABLE 2c
List of odorant compounds.

| Compound Name | CAS # |
|---|---|
| Abhexone | 698-10-2 |
| Acetophenone | 98-86-2 |
| AcetylPyridine | 1122-62-9 |
| Adoxal | 141-13-9 |
| AldehydeC-16highcon | 77-83-8 |
| AldehydeC-16lowcon | 77-83-8 |
| AldehydeC-18 | 104-61-0 |
| AllylCaproate | 123-68-2 |
| AmylAcetateiso-amylAcetat | 123-92-2 |
| AmylButyrate | 540-18-1 |
| AmylCinnamicAldehyeDiethy | 60763-41-9 |
| AmylPhenylAcetate | 102-19-2 |
| AmylValerate | 2173-56-0 |
| Andrane | 29597-36-2 |
| Anethole | 104-46-1 |
| Anisole | 100-66-3 |
| Auralva | 89-43-0 |
| Benzaldehyde | 100-52-7 |
| BenzoDihydropyrone | 119-84-6 |
| BornylAcetateiso-BornylA | 5655-61-8 |
| ButanoicAcid | 107-92-6 |
| Butanol | 71-36-3 |
| ButylQuinolineiso | 544-40-1 |
| ButylSulfide | 67634-06-4 |
| Camphordl | 464-48-2 |
| Carvone-1 | 99-49-0 |
| Caryophyllene | 87-44-5 |
| Cashmeran | 33704-61-9 |
| Celeriax | 17369-59-4 |
| Chlorothymol | 89-68-9 |
| CinnamicAldehyde | 104-55-2 |
| Citral | 141-27-5 |
| Citralva | 5585-39-7 |
| Coumarin | 91-64-5 |
| Cresol-m | 108-39-4 |
| Cresol-p | 106-44-5 |
| CresylAcetate-p | 140-39-6 |
| CresylMethylEther-p | 103-93-5 |
| CresylisoButyrate-p | 104-93-8 |
| CuminicAldehyde | 122-03-2 |
| Cyclocitral-iso | 1423-46-7 |
| Cyclodithalfarol | 55704-78-4 |
| Cyclohexanedione1,2 | 765-87-7 |
| Cyclohexanol | 108-93-0 |
| Cyclotene | 80-71-7 |
| Cyclotropal | 67634-23-5 |
| Decad ienal2,4-trans-trans | 25152-84-5 |
| DecahydroNaphthalene | 91-17-8 |
| DibutylAmine | 111-92-2 |
| DiethylSulfide | 352-93-2 |
| DimethylBenzylCarbinylBut | 10094-34-5 |
| DimethylPhenylEthylCarb | 103-05-9 |
| DimethylPyrazine2,3 | 5910-89-4 |
| DimethylPyrazine2,5 | 123-32-0 |
| DimethylPyrrole2,5 | 625-84-3 |
| DimethylTrisulfide | 3658-80-8 |
| Diola | 7/3/7474 |
| EthylPropionate | 105-37-3 |
| EthylPyrazinehighconc | 13925-00-3 |
| EthylPyrazinelowconc | 13925-00-3 |
| Eucalyptol | 470-82-6 |
| Floralozone | 67634-15-5 |
| Furfural | 98-01-1 |
| FurfurylMercaptan | 98-02-2 |
| Heptanoll | 111-70-6 |
| Hexanal | 68-25-1 |
| Hexanol1 | 111-27-3 |
| Hexanol3 | 623-37-0 |
| Hexenal-trans1 | 6728-26-3 |
| HexylArninehighconc | 111-26-2 |
| HexylArninelowconc | 111-26-2 |
| HexylCinnamicAldehyde | 101-86-0 |
| HydratropicAldehydeDiAl | 90-87-9 |
| HydroxyCitronellal | 107-75-5 |
| Indole | 120-72-9 |
| Indolene | 67801-36-9 |
| Iodoform | 75-47-8 |
| Ionone-betahig hconc | 14901-07-6 |
| Ionone-betalowconc | 14901-07-6 |
| Ironealpha | 79-69-6 |
| Limonened | 126-91-0 |
| Linalool | 138-86-3 |
| Lyral | 31906-04-4 |
| Maritima | 67258-87-1 |
| Melonal | 106-72-9 |
| Menthol-1 | 2216-51-5 |
| MethoxyNaphthalene2 | 93-04-9 |
| Methyl-iso-Borneol2 | 134-20-3 |
| Methyl-iso-Nicotinate | 462-95-3 |
| MethylAcetaldehydeDiAce | 611-13-2 |
| MethylAnthranilate | 2271-428 |
| MethylFuroate | 491-35-0 |
| MethylQuinolinepara | 2459-09-8 |
| MethylThiobutyrate | 68917-50-50 |
| Methylsalicylate | 2432-51-1 |
| MuskGalaxolide | 1222-05-5 |
| MuskTonalid | 1508-02-1 |
| Myracaldehyde | 37677-14-8 |
| NonylAcetate | 143-13-5 |
| Nootkatone | 4674-50-4 |
| Octanol1 | 111-87-5 |
| Octenol-1-3-OL | 3391-86-4 |
| PentanoicAcid | 109-52-4 |
| PentenoicAcid4 | 591-80-0 |
| PhenylAceticAcid | 103-82-2 |
| PhenylAcetylene | 536-74-3 |
| PhenylEthanolhighconc | 60-12-8 |
| PhenylEthanollowconc | 60-12-8 |
| Phorone-iso | 78-59-1 |
| Pinenealpha | 80-56-8 |
| PropylButyrate | 105-66-8 |
| PropylQuinoline-iso | 135-79-5 |
| PropylSulfide | 111-47-7 |
| Pyridine | 110-86-1 |
| Safrole | 94-59-7 |
| Sandiff | 69460-08-8 |
| Santalol | 115-71-9 |
| Skatole | 83-34-1 |
| Terpineolmostlyalpha | 10482-56-1 |
| TetrahydroThiophene | 110-01-0 |
| Tetraquinone | 91-61-2 |
| Thienopyrimidine | 3626-71-7 |
| ThioglycolicAcid | 123-93-3 |
| Thiophene | 110-02-1 |
| Thymol | 89-83-8 |
| Tolualdehyde-ortho | 529-20-4 |
| Toluenehighconc | 108-88-3 |
| Toluenelowconc | 108-88-3 |
| TrimethylAmine | 75-50-3 |
| Undecalactonegamma | 104-67-6 |
| UndecylenicAcid | 112-38-9 |
| Valeraldehyde-iso | 590-86-3 |
| ValericAcid-iso | 503-74-2 |
| Valerolactonegamma | 108-29-2 |
| Vanillin | 121-33-5 |
| Zingerone | 122-48-5 |

Sensory stimulus data also includes correlation of a substance, such as a beverage or a foodstuff, with the olfactory receptor or receptors that are stimulated by the substance; and/or with the olfactory receptors that are not stimulated by the substance.

Sensory stimulus data also includes identification of chemical compounds (and combinations thereof) that produce particular tastes. Accordingly, sensory stimulus data also includes identification of one or a plurality of gustatory (taste) receptors that are stimulated by a particular chemical compound or combination of compounds. Alternatively, or in addition, sensory stimulus data includes identification of one or a plurality of gustatory receptors that are not stimulated by a particular chemical compound or combination of compounds. Sensory stimulus data also includes correlation of a substance, such as a beverage or a foodstuff, with the gustatory receptor or receptors that are stimulated by the substance; and/or with the gustatory receptors that are not stimulated by the substance. Gustatory receptors provide the basic sensations of sweet, sour, salty, bitter and umami. Additional taste sensations include astringent and pungent.

Databases of reference stimuli (e.g., odors), and their corresponding emotional responses, can be compiled. For example, a subject is exposed to the reference odor C and is asked to rate his/her happiness in response to the smell of the reference odor C on a scale of 1-10. Multiple subjects are tested with the reference odor C and the average happiness level for odor C is 5. The same is done for reference odor D, and the average happiness level for odor D is determined to be 9.

A database of reference odors and their corresponding emotional states (e.g., happiness in this case) can be built using this method. Additional attributes can be included in the database. For example, a sub-group of subjects in U.S. may rate the reference odor D to have an average happiness of 9.5, while another sub-group of subjects in Europe may rate the reference odor D to have an average happiness of 8.5. Therefore, based on the additional attribute (e.g., geolocation, nationality, gender, age, and so on), the reference odors and its corresponding emotional state for specific groups of subjects can be obtained and stored in the database.

5. Emotional Response Data

An “emotional response” refers to the reaction of a subject to a particular sensory stimulus. An emotional response is characterized by one or both of objective data and subjective data.

Objective data include physical and physiological reactions such as, for example, facial expressions, micro expressions, brain signals, electroencephalography (EEG) signals, functional magnetic resonance imaging (fMRI) signals, production of body chemicals (e.g., hormones, cytokines), pupil dilation, skin conductance, skin potential, skin resistance, skin temperature, respiratory frequency, blood pressure, blood flow, saliva production and flow rate, and any combination thereof.

Subjective data include feeling experienced by the subject when exposed to the sensory stimulus. Such feelings can be positive, negative or neutral. Positive feelings include, for example, amused, blissful, calm, cheerful, content, dreamy, ecstatic, energetic, excited, flirty, giddy, good, happy, joyful, loving, mellow, optimistic, peaceful, silly, and sympathetic. Negative feelings include, for example, angry, annoyed, apathetic, bad, cranky, depressed, envious, frustrated, gloomy, grumpy, guilty, irritated, melancholy, pessimistic, rejected, restless, sad, stressed, and weird. Neutral feelings can include, for example, indifference.

In creating the learning algorithms disclosed herein, a subject is exposed to a sensory stimulus (i.e., a smell or a taste), and the emotional response of the subject, to the sensory stimulus, is assessed. An emotional response can be objective or subjective; and both objective and subjective emotional responses are used to populate the database used to generate the learning algorithm.

a) Exposure to Sensory Stimulus

In order to collect sensory response data a subject can be seated in a room. Subjects can be exposed to an odor by, for example, filling the room with the odor or placing a carrier from which the odor permanent permeates, for example a swab or vial, under the subject's nose. In the case of taste, substance containing taste can be placed into the subject's mouth or swabbed on the subject's tongue. The substance can be a solid or liquid. It could have texture or no texture. It could be food or a drink.

Upon exposure to the sensory stimulus, emotional response data can be collected from the subject. Emotional response data can be, for example, objective data which can be measured by an operator. Accordingly, the subject can be monitored using various tools as described herein. Also, response can be a subjective response. A subjective response is one that cannot be measured by a third-party but must be communicated by the subject, as described herein.

b) Qualifying an Emotional Response

In certain embodiments, each subject for which information is obtained is exposed to an odor or taste, and the physiological and/or emotional responses of the subject, to that particular odor, are determined, e.g., as a quantitative or qualitative measurement. Sensory stimulus data, for use in populating databases as described herein, can comprise data on at least 2, 5, 10, 25, 50, 75, 100, 200, 300, 400, 500 (and any integral value therebetween) or more different sensory stimuli.

Emotional response can be classified as belonging to any of a number of different discrete categories, such as, for example, anger, joy, sadness, fear and disgust. Within each category, the emotional response can be further characterized as binary (e.g., present or not present) or on a continuous or discrete scale indicating intensity of the emotion. Such a scale can be numeric, e.g., ranging from 1 to 10, or descriptive. For example, an angry emotional response could be characterized as present or absent, on a scale of low-to-high in which one is low into 10 is high or, linguistically described as annoyed, angry or enraged.

For instance, a given emotional response (e.g., happiness) can be ranked on a numerical scale (e.g., 1 to 10) ranging from the least positive to the most positive response for the particular emotion under study. Thus, in certain embodiments, an emotional response is classified into a category within a set of discrete categories, wherein the discrete categories are hierarchically arranged from the least positive to the most positive emotional response. The set of categories can contain any number of discrete categories (e.g., 2, 3, 4, 5, 6, 7, 8, 9, 10 or more). In further embodiments, the emotional response is classified as a number (e.g., 1 to 10), a degree (e.g., mild, neutral, severe/intense), a level (e.g., weak, strong) a range (e.g., low, medium, high) or a bucket.

Another means by which a subject can report an emotional response is by classifying the response into a category within a set of discrete categories, wherein the discrete categories are hierarchically arranged from the least positive emotional response to the most positive emotional response. The set of discrete categories can contain any of 2, 3, 4, 5, 6, 7, 8, 9, 10 or more discrete categories. In one embodiment, the set includes two discrete categories: a negative emotional response and a positive emotional response.

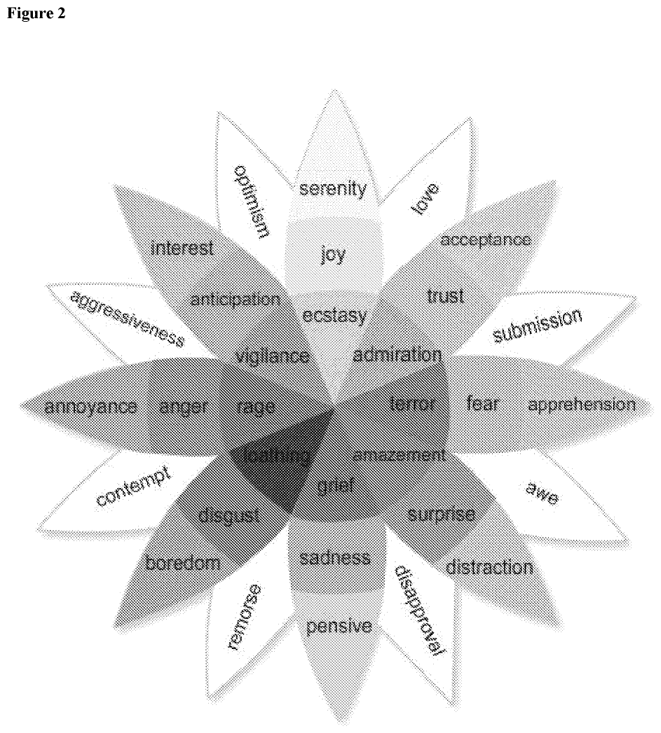

An emotional response can also be classified in multiple emotional dimensions as a multi-variate response in which a plurality of different feelings are assessed. In certain embodiments, each feeling is measured on a scale. For example, a subject can describe feelings of relative happiness on a scale of 1 to 10, in which 1 is miserable and 10 is overjoyed. Exemplary variables (e.g., feelings) include one or a plurality of love, submission, awe, disapproval, remorse, contempt, aggressiveness and optimism. See also FIG. 2.

Classification of an emotional state can be derived by a combination of subjective and objective responses. For example, classifying an individual as being in a state of rage can depend upon a person subjective response (e.g., “I'm really angry!”) as well as objective responses (increased heart rate, flushing of the skin, tensing of the muscles). Use of both subjective and objective data in classifying an emotional response can reduce differences between individuals who may linguistically describe the same response in different terms. Accordingly, in developing a classifier, a machine learning algorithm may treat the collection of subjective and objective responses as the categorical variable, or, may simply classify based on a single subjective response.

c) Subjective Response Data

Subjective emotional responses include feelings experienced by the subject when exposed to the sensory stimulus. Such feelings can be positive, negative or neutral. Positive feelings include, for example, amused, blissful, calm, cheerful, content, dreamy, ecstatic, energetic, excited, flirty, giddy, good, happy, joyful, loving, mellow, optimistic, peaceful, silly, and sympathetic. Negative feelings include, for example, angry, annoyed, apathetic, bad, cranky, depressed, envious, frustrated, gloomy, grumpy, guilty, irritated, melancholy, pessimistic, rejected, restless, sad, stressed, and weird. Neutral feelings can include, for example, indifference.

There are a number of ways in which a subjective emotional response can be conveyed. For example, the subject can be asked to describe her or his emotional state, either verbally or in writing. A subjective response can also include a linguistic expression of the subject such as a spoken (oral) response, a written response or a signed (i.e., conveyed by sign language) response.

In other embodiments, a subject can be shown a plurality of images, and asked to select the image which most closely corresponds to his or her emotional state. The subject can rank his or her emotional response on a numerical scale. Images can include, for example, pictures of people with different facial expressions.

The linguistic expression may be descriptors of the sensory stimulus. The descriptors can comprise, but are not limited to, fruity, sweet, perfumery, aromatic, floral, rose, spicy, cologne, cherry, incense, orange, lavender, clove, strawberry, anise, violets, grape juice, pineapple, almond, vanilla, peach fruit, honey, pear, sickening, rancid, sour, vinegar, sulfidic, dirty linen, urine, green pepper, celery, maple syrup, caramel, woody, coconut, soupy, burnt milk, eggy, apple, light, musk, leather, wet wool, raw cucumber, chocolate, banana, coffee, yeasty, cheesy, sooty, blood, raw meat, fishy, bitter, clove, peanut butter, metallic, tea leaves, stale, mouse, seminal, dill, molasses, cinnamon, heavy, popcorn, kerosene, fecal, alcoholic, cleaning fluid, gasoline, sharp, raisins, onion, buttery, and herbal.

In some cases, the emotional state of the subject can be assigned to a grading scale. For example, the subject can be asked to choose an option (1 to 9) on the following grading scale when given a testing substance (e.g., a beverage, such as water):

1) I would be very happy to accept this water as my everyday drinking water;

2) I would be happy to accept this water as my everyday drinking water;

3) I am sure that I could accept this water as my everyday drinking water;

4) I could accept this water as my everyday drinking water;

5) Maybe I could accept this water as my everyday drinking water;

6) I don't think I could accept this water as my everyday drinking water;

7) I could not accept this water as my every day drinking water;

8) I could never drink this water;

9) I can't stand this water in my mouth and I could never drink it.

Linguistic expressions of the subject can be recorded and analyzed for assessing the physiological state of the subject. The linguistic expression can be any physical form (e.g., sound, visual image or sequence thereof) used to represent a linguistic unit. The linguistic expression can be spoken, written, or signed. The linguistic expression can be translated into text (e.g., using a computer algorithm). The linguistic expression can be classified into an emotional state such as happiness, surprise, anger, fear, sadness, or disgust. In some cases, the subjects can be asked to give their emotional states. In some cases, the subjects can be asked to assign their emotional states to one or more images associated with the emotional states. In some cases, the subjects can be given a list of words to formulate their emotional states, thereby mapping the linguistic expressions to the emotional states in a more restricted way.

For the analysis of the linguistic expressions, a computer algorithm (e.g., machine learning algorithm) can extract features from the voice (e.g., tone) and/or from the content.

The sensors can be used to detect and/or measure physiological signals of the subject that is reacting to different stimulus associated with targeted emotions.

Classical stimuli, such as music, images, movie scenes, and video games can be used to train the computer algorithm to make the correct connection between the physiological signals when given classical stimuli and the corresponding classical emotions (e.g., happiness, sadness). For example, images known to elicit happiness can be given to the subjects, and then the physiological signals measured from the subject can be linked to the target emotional state, e.g., happiness.

Synesketch algorithms can be used to analyze emotional content of text sentences in terms of emotional types (e.g., happiness, sadness, anger, fear, disgust, and surprise), weights (how intense the emotion is), and/or a valence (is it positive or negative). The recognition technique can be grounded on a refined keyword spotting method which can employ a set of heuristic rules, a WordNet-based word lexicon, and/or a lexicon of emoticons and common abbreviations.

Articles linked with classical emotions (e.g., happiness, sadness), but also emotions more taste and/or smell related can be used. These articles can be taken from database (for comparison with similar studies) for classical emotions and can be used to generate taste and/or smell related emotions.

Evaluation can be made on base compounds. The base compound can be a smelling and/or tasting reference compound with expected results. For example, sweet reference compound can be expected to be associated with joy. Evaluation can also be made on compounds with unknown results.

A subjective response can also be inferred by the activity of the subject on social media; for example, whether or not a subject posts information relating to an experience with a sensory stimulus. Subjects may post or otherwise be active on social media. Such activity can indicate a subject's reaction to sensory stimuli of various products. For example, social media data may indicate changes in spending patterns with respect to a product that contains a sensory stimulus. It may also contain posts including comments or rankings about such products or sensory stimuli. Such reactions may change over time. Furthermore, data also can be scraped from persons sharing group status or identity with a subject, such as ethnicity, socio-economic status, sex/gender, geographic region, religion, etc. Such data can be included among the emotional response data. Therefore, reactions on social media provide updated indications of emotional reaction to the same sensory stimulus. Social media include, without limitation, social networks, media sharing networks, discussion forums, bookmarking and content curation networks, consumer review networks, blogging and publishing networks, interest-based networks, social shopping networks, sharing economy networks and anonymous social networks.

d) Objective Response Data

An objective (or physiological) emotional response is one that can generally be measured and quantitated. Objective data include physical and physiological reactions such as, for example, facial expressions, micro expressions, brain signals, electroencephalography (EEG) signals, cardiac signals (e.g., EKG, pulse rate), functional magnetic resonance imaging (fMRI) signals, body chemical stimuli, production of body chemicals (e.g., hormones, cytokines), pupil dilation, skin conductance, skin potential, skin resistance, skin temperature, respiratory frequency, blood pressure, blood flow, saliva production and flow rate, and any combination thereof.

Certain emotional responses, primarily objective responses, can be quantitated using an instrument or device appropriate to the response being measured. In other cases, often in the case of a subjective response, the subject will provide a measure of the intensity of the response. For example, an emotional response can be a simple binary response (e.g., yes/no, like/dislike, happy/sad) or an emotional response can be classified as part of a range, either a discrete range or a continuous range. An emotional response can be classified as, for example a number, a degree, a level, a range or a bucket. An emotional response can be a subjective feeling that is communicated by the subject verbally, in writing or in sign language. In additional embodiments, an emotional response is classified as an image, either selected by the subject from a group of images or created by the subject, for example, by a drawing.

In certain embodiments, the method further comprises analyzing a reference physiological signal from the subject in response to a reference odor or taste. In some cases, the reference odor or taste elicits a reference physiological state. In some cases, the method further comprises comparing the physiological signal from the subject with the reference odor or taste. In some cases, the method further comprises assigning the physiological signal from the subject to the reference odor or taste, wherein the physiological signal from the subject is comparable to reference odor or taste. In some cases, the physiological signal from the subject is within ±50%, ±40%, ±30%, ±20%, ±10%, ±5%, ±2%, or ±1% of the reference physiological signal.

The method for assessing a physiological state of a subject in response to a stimulus can comprise analyzing a physiological signal from the subject. The physiological signal can be detected using a sensor. The physiological signal can be facial expressions, micro expressions, brain signals, electroencephalography (EEG) signals, functional magnetic resonance imaging (fMRI) signals, body odors, pupil dilation, skin conductance, skin potential, skin resistance, skin temperature, respiratory frequency, blood pressure, blood flow, saliva, or any combination thereof. These human emotional state markers can pick up different signal modalities from specific human organs which can yield a large amount of information about the emotional state of person, from happy to sad, with hundreds of shades between.

The method can further comprise characterizing the physiological state of the subject using the analyzed information, for instance, using a machine learning algorithm. Several machine learning algorithms can be used as emotion classifiers such as Support Vector Machine (SVM), Naïve Bayes (NB), Quadratic Discriminant Analysis (QDA), K-Nearest Neighbors (KNN), Linear Discriminant Analysis (LDA), and Multilayer Perceptron (MLP).

Facial expressions can be obtained by an image-capturing sensor, such as a camera. Facial expressions can be obtained from static images, image sequences, or video. Facial expressions can be analyzed using geometric-based approaches or appearance-based approaches. Geometric-based approaches, such as active shape model (ASM), can track the facial geometry information over time and classify expressions based on the deformation. Appearance-based approaches can describe the appearance of facial features and/or their dynamics.

In some cases, analyzing facial expressions can comprise aligning the face images (to compensate for large global motion and maintain facial feature motion detail). In some cases, analyzing facial expressions can comprise generating an avatar reference face model (e.g., Emotion Avatar Image (EAI) as a single good representation) onto which each face image is aligned to (e.g., using an iterative algorithm). In some cases, analyzing facial expressions can comprise extracting features from avatar reference face model (e.g., using Local Binary Pattern (LBP) and/or Local Phase Quantization (LPQ)). In some cases, analyzing facial expressions can comprise categorizing the avatar reference face model into a physiological state using a classifier, such as the linear kernel support vector machines (SVM).

Facial expressions, including micro expressions, can be detected using the facial action coding system (FACS). FACS can identify the muscles that produce the facial expressions and measure the muscle movements using the action unit (AU). FACS can measure the relaxation or contraction of each individual muscle and assigns a unit. One or more muscle can be grouped into an AUs. Similarly, one muscle can be divided into separate AUs. FACS can assign a score consists of duration, intensity, and/or asymmetry.

EEG, the signal from voltage fluctuations in the brain, can be used for assessing the physiological state of the subject. Emotion can be related with some structures in the center of the brain including limbic system, which includes amygdala, thalamus, hypothalamus, and hippocampus. EEG can be obtained by recording the electrical activity on the scalp using a sensor (e.g., electrode). EEG can measure voltage changes resulting from ionic current flows within the neurons of the brain. EEG can measure five major brain waves distinguished by their different frequency bands (number of waves per second), from low to high frequencies, respectively, called Delta (1-3 Hz), Theta (4-7 Hz), Alpha (8-13 Hz), Beta (14-30 Hz), and Gamma (31-50 Hz).

fMRI can be used for assessing the physiological state of the subject. fMRI can measure brain activity by detecting changes associated with blood flow. fMRI can use the blood-oxygen-level dependent (BOLD) contrast. Neural activity in the brain can be detected using a brain or body scan by imaging the change in blood flow (hemodynamic response) related to energy use by brain cells. fMRI can use arterial spin labeling and/or diffusion magnetic resonance imaging MRI.

Skin conditions, such as skin conductance, skin potential, skin resistance, and skin temperature can be detected and measured using electronic sensors. For example, skin conductance can be detected and measured using an EDA meter, a device that displays the change electrical conductance between two points over time. In another example, galvanic skin response can be detected and measured using a polygraph device.

In certain embodiments, a baseline response (e.g., a response in the absence of the sensory stimulus) is determined prior to assessing an emotional response of a subject. Baselines can be established for both subjective and objective emotional responses. In one embodiment, measurement of a baseline comprises exposing the subject to a neutral stimulus, such as the taste or odor of water, a breath of pure gas, such as oxygen or nitrogen, or to a calming environment, such as might be produced by dim lighting or quiet music.

e) Methods of Collecting Data

In some cases, the emotional state of the subject can be classified using a computer algorithm. The emotional state can be further classified into one or more levels. For example, an emotional state (e.g., happiness) can be further classified into 10 numeric levels (e.g., 1 being the lowest happiness level and 10 being the highest happiness level).

Preparation: Human subjects can be individually surveyed (to not influence each other). A number of external parameters, such as position of the subject, temperature of the room, light in the room, sound in the room (no background sound), can be maintained at a constant level to cancel body signal variations coming from senses other than taste and/or smell. In some cases, the subject can perform a meditation, eat a meal, and/or take a shower under controlled conditions to cancel body signal variations.

Baseline measurement: Physiological signals can be detected and/or measured from the non-stimulated subject in order to have a baseline before stimulus. In some cases, the subject can take a control substance (e.g., air or water) to assess the subject's physiological state without the inducement of the stimulus.

Emotions reference measurement: The sensors can be used to detect and/or measure physiological signals of the subject that are reactive to different stimulus associated with targeted emotions.

Compounds responses: Evaluation can be made on base compounds. The base compound can be a smelling and/or tasting reference compound with expected results. For example, sweet reference compound can be expected to be associated with joy. Evaluation can also be made on compounds with unknown results.

Features extraction and features engineering: Different features can be extracted from the physiological signals. These features can be engineered (e.g., remove baseline) and used as input to a computer algorithm, such as a machine learning algorithm, to match these features with the compounds.

B. Training Machine Learning Algorithms

1. Machine Learning Algorithms

Machine learning refers to any of a variety of machine learning algorithms known to those of skill in the art are suitable for use in the methods described herein. Examples include algorithms, unsupervised learning algorithms, semi-supervised learning algorithms, reinforcement learning algorithms, deep learning algorithms, or any combination thereof.

Machine learning algorithms (e.g. artificial intelligence algorithms) can be selected from support vector machine (SVM), naïve bayes (NB), quadratic discriminant analysis (QDA), linear discriminant analysis (LDA), multilayer perceptron (MLP), artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors (KNN)), and ensemble methods (e.g., bagging, boosting), naive bayes, k-means clustering, dimensionality reduction algorithms, gradient boosting algorithms, such as gradient boosting machine (GBM), extreme gradient boosting (XGBoost), LightGBM, and CatBoost, or any combination thereof. In certain embodiments, machine learning is unsupervised and emotional responses are classified into one of a plurality of clusters based on both subjective responses and objective responses.

Supervised learning algorithms: In the context of the present disclosure, supervised learning algorithms are algorithms that rely on the use of a set of linked training data examples (e.g., sets of subject profile, sensory stimulus and the corresponding emotional response(s)) to infer the relationship between sensory stimulus and emotional response for a given subject profile.

Unsupervised learning algorithms: In the context of the present disclosure, unsupervised learning algorithms are algorithms used to draw inferences from training data sets consisting of sensor signal patterns that are not linked. The most commonly used unsupervised learning algorithm is cluster analysis, which is often used for exploratory data analysis to find hidden patterns or groupings in process data.

Semi-supervised learning algorithms: In the context of the present disclosure, semi-supervised learning algorithms are algorithms that make use of both labeled and unlabeled data for training (typically using a relatively small amount of labeled data with a large amount of unlabeled data).

Reinforcement learning algorithms: Reinforcement learning algorithms are commonly used for optimizing Markov decision processes (i.e., mathematical models used for studying a wide range of optimization problems where future behavior cannot be accurately predicted from past behavior alone, but rather also depends on random chance or probability). Q-learning is an example of a class of reinforcement learning algorithms. Reinforcement learning algorithms differ from supervised learning algorithms in that correct training data input/output pairs are never presented, nor are sub-optimal actions explicitly corrected. These algorithms tend to be implemented with a focus on real-time performance through finding a balance between exploration of possible outcomes (e.g., emotional response identification) based on updated input data and exploitation of past training.

Deep learning algorithms: In the context of the present disclosure, deep learning algorithms are algorithms inspired by the structure and function of the human brain called artificial neural networks (ANNs), and specifically large neural networks comprising multiple hidden layers, that are used to map an input data set (e.g. a subject profile) to, for example, an emotional response.

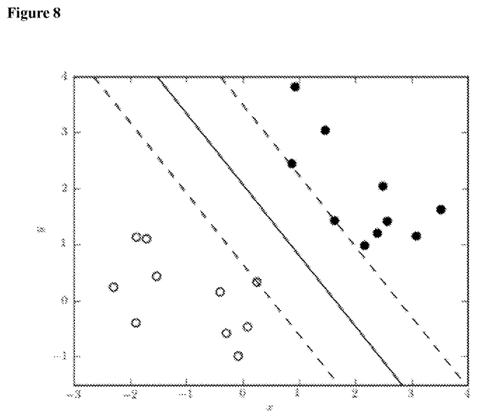

Support vector machine learning algorithms: Support vector machines (SVMs) are supervised learning algorithms that analyze data used for classification and regression analysis. Given a set of training data examples, each marked as belonging to one or the other of two categories, an SVM training algorithm builds a linear or non-linear classifier model that assigns new data examples to one category or the other. (FIG. 8.)

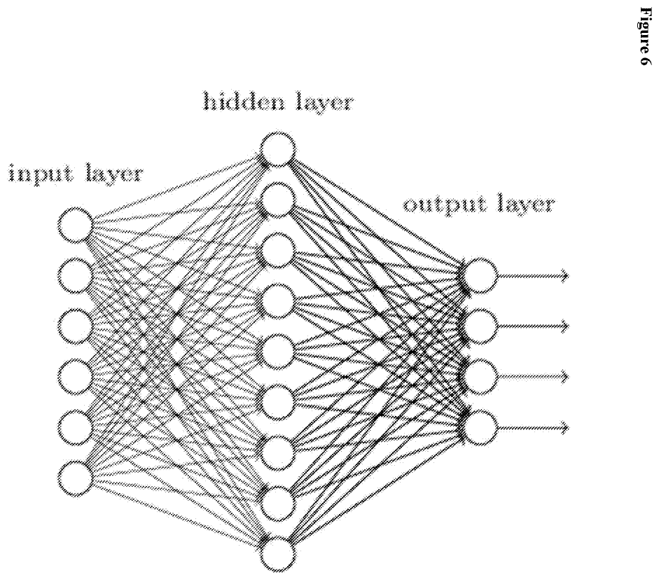

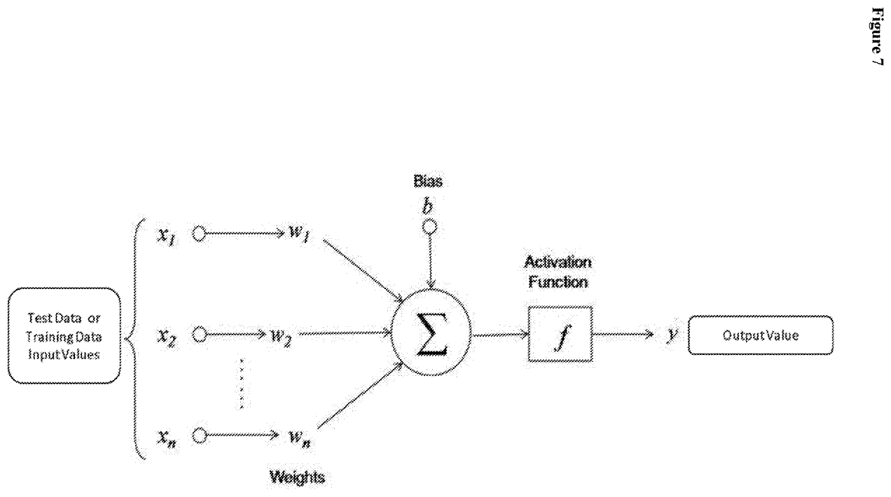

Artificial neural networks & deep learning algorithms: Artificial neural networks (ANN) are machine learning algorithms that can be trained to map an input data set (e.g., sensory stimuli) to an output data set (e.g., emotional responses), where the ANN comprises an interconnected group of nodes organized into multiple layers of nodes (FIG. 6). For example, the ANN architecture can comprise at least an input layer, one or more hidden layers, and an output layer. The ANN can comprise any total number of layers, and any number of hidden layers, in which the hidden layers function as trainable feature extractors that allow mapping of a set of input data to an output value or set of output values. As used herein, a deep learning algorithm (DNN) is an ANN comprising a plurality of hidden layers, e.g., two or more hidden layers. Each layer of the neural network comprises a number of nodes (or “neurons”). A node receives input that comes either directly from the input data (e.g., sensory stimuli) or the output of nodes in previous layers, and performs a specific operation, e.g., a summation operation. In some cases, a connection from an input to a node is associated with a weight (or weighting factor). In some cases, the node may sum up the products of all pairs of inputs, xi, and their associated weights (FIG. 7). In some cases, the weighted sum is offset with a bias, b, as illustrated in FIG. 6. In some cases, the output of a node or neuron is gated using a threshold or activation function, f, which can be a linear or non-linear function. The activation function can be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training data. For example, the parameters can be trained using the input data from a training data set and a gradient descent or backward propagation method so that the output value(s) (e.g., a determination of emotional response) that the ANN computes are consistent with the examples included in the training data set. The parameters can be obtained from a back propagation neural network training process that may or may not be performed using the same computer system hardware as that used for performing the cell-based sensor signal processing methods disclosed herein.

Any of a variety of neural networks known to those of skill in the art are suitable for use in the methods and systems of the present disclosure. Examples include, but are not limited to, feedforward neural networks, radial basis function networks, recurrent neural networks, convolutional neural networks, and the like. In some embodiments, the disclosed sensor signal processing methods can employ a pretrained ANN or deep learning architecture. In some embodiments, the disclosed sensor signal processing methods may employ an ANN or deep learning architecture wherein the training data set is continuously updated with real-time data.

In general, the number of nodes used in the input layer of the ANN or DNN can range from about 10 to about 100,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, or at least 100,000. In some instances, the number of node used in the input layer may be at most 100,000, at most 90,000, at most 80,000, at most 70,000, at most 60,000, at most 50,000, at most 40,000, at most 30,000, at most 20,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. Those of skill in the art will recognize that the number of nodes used in the input layer can have any value within this range, for example, about 512 nodes.

In some instance, the total number of layers used in the ANN or DNN (including input and output layers) ranges from about 3 to about 20. In some instances, the total number of layers is at least 3, at least 4, at least 5, at least 10, at least 15, or at least 20. In some instances, the total number of layers is at most 20, at most 15, at most 10, at most 5, at most 4, or at most 3. Those of skill in the art will recognize that the total number of layers used in the ANN can have any value within this range, for example, 8 layers.

In some instances, the total number of learnable or trainable parameters, e.g., weighting factors, biases, or threshold values, used in the ANN or DNN ranges from about 1 to about 10,000. In some instances, the total number of learnable parameters is at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters is any number less than 100, any number between 100 and 10,000, or a number greater than 10,000. In some instances, the total number of learnable parameters is at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Those of skill in the art will recognize that the total number of learnable parameters used can have any value within this range, for example, about 2,200 parameters.

Distributed data processing systems and cloud-based training databases: In some embodiments, the machine learning-based methods disclosed herein are used for processing data on one or more computer systems that reside at a single physical/geographical location. In other embodiments, they may be deployed as part of a distributed system of computers that comprises two or more computer systems residing at two or more physical/geographical locations. Different computer systems, or components or modules thereof, may be physically located in different workspaces and/or worksites (i.e., in different physical/geographical locations), and may be linked via a local area network (LAN), an intranet, an extranet, or the internet so that data to be processed may be shared and exchanged between the sites.

In some embodiments, training data resides in a cloud-based database that is accessible from local and/or remote computer systems on which the machine learning-based sensor signal processing algorithms are running. As used herein, the term “cloud-based” refers to shared or sharable storage of electronic data. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site. In some embodiments, test results generated locally can be uploaded to a cloud-based database and used to update the training data set in real time for continuous improvement of system test performance.

2. Training

Training a learning algorithm on a dataset as described herein produces one or a plurality of classification algorithms which will infer a class of emotional response to a sensory stimulus based on subject profile data. An operator can select from among classifiers generated based on parameters such as sensitivity, specificity, positive predictive value, negative predictive value or receiver operating characteristics such as area under the curve. The classifier may rely more heavily on certain traits in a subject profile then others in making the inference. Accordingly, when executed in the classifier it may suffice to provide a subject profile that includes only those traits needed by the classifier to make an inference.

Emotional response may be based on a single subject response, such as a verbal indication of emotional state. However, objective measurements also can inform a subject's emotional state. Therefore, a classifier may group cluster combinations of subjective and objective responses, or even objective responses alone as defining an emotional response or in grading an emotional response. So, for example, a degree of anxiety may be based on both a verbal response of anxiety as well as physiological responses such as increased heart rate, and increase sweating.

In certain embodiments, the classification algorithms disclosed herein are used to predict emotional responses of a subject who is in contact with a compound or a mixture of compounds. The process of predicting physiological states (e.g., emotional responses) of the subject can be conducted after mapping physiological states to a human olfactory receptor (hOR) or to a combination of hORs.

To predict physiological states, one or more algorithms may be used. The one or more algorithms may be machine learning algorithms. The one or more algorithms may be associated with statistical techniques. The one or more statistical techniques may include principal component analysis. The principal component analysis may comprise reducing the dimensionality of perceptual descriptors of the sensory stimulus. The dimensionality of perceptual descriptors may be the number of perceptual descriptors. The number of physicochemical descriptors may be at least 1, 5, 10, 50, 100, 200, 300, 400, 500, 600, 700, 800, 900, 1000, 1100, 1200, 1300, 1400, 1500, 1600, 1700, 1800, or greater. The dimensionality of perceptual descriptors may be reduced to one perceptual principal component. The perceptual principal component may be pleasantness or happiness. The pleasantness or happiness may refer to the continuum from unpleasant to pleasant.

The principal component analysis may comprise reducing the dimensionality of physicochemical descriptors of a compound or compounds serving as the sensory stimulus. The dimensionality of physicochemical descriptors may be the number of physicochemical descriptors. The number of physicochemical descriptors may be at least 1, 5, 10, 50, 100, 200, 300, 400, 500, 600, 700, 800, 900, 1000, 1100, 1200, 1300, 1400, 1500, 1600, 1700, 1800, or greater. The physicochemical descriptors may describe the molecular features of the compound or compounds. The physicochemical descriptors include, but are not limited to, the carbon atom number, the molecular weight, the number of carbon-carbon bonds, the number of functional groups, the aromaticity index, the maximal electrotopological negative variation, the number of benzene-like rings, the number of aromatic hydroxyls, the average span R, the number of carboxylic acid groups, and the number of double bonds. The dimensionality of perceptual descriptors may be reduced to one physicochemical principal component. The physicochemical principal component may be a sum of atomic van der Waals volumes.

The principal component analysis may further comprise finding that perceptual principal component may have a privileged link to physicochemical principal component. The privileged link may be linear relationship between the perceptual principal component and physicochemical principal component. The privileged link may allow a single optimal axis for explaining the variance in the physicochemical data to be the best predictor of perceptual data. Predicted physiological states can be used in situations such as malodorant blocker, culturally targeted product design, harmful chemicals detection, or triggering specific targeted emotions.

The following steps are executed to predict physiological states of subjects.

The machine leaning algorithm can comprise linear regression, logistic regression, decision tree, support vector machines (SVM), naive bayes, k-nearest neighbors algorithm (k-NN), k-means clustering, random forest, dimensionality reduction algorithms, gradient boosting algorithms, such as gradient boosting machine (GBM), extreme gradient boosting (XGBoost), LightGBM, and CatBoost, or any combination thereof.

The combination of data sets with the presentation of taste, smell, sound, images and/or tactile signal can be used to predict a subject's emotional state (e.g., happiness or sadness). The methods can be used to design a set of optimal stimuli to provide a desired response.

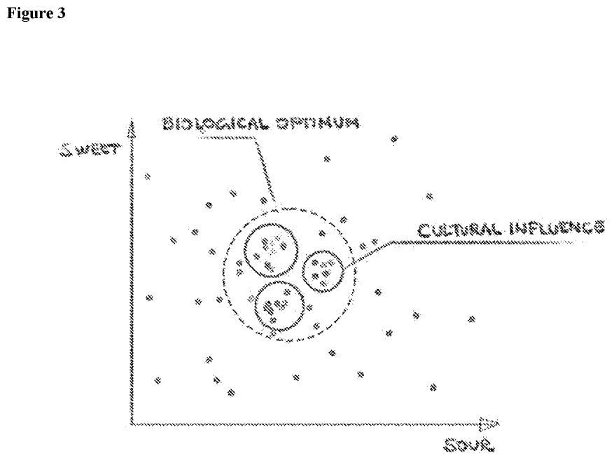

The method can be used for the creation of a precise emotions flower for general emotions (as shown in FIG. 2) and/or for smell/taste related emotions. The method can be used to map between a selected database of sensory stimuli (e.g., compounds) and their corresponding emotions. The method can be applied to different groups of people, with groups selected on the basis of, for example, ethnicity, culture, and/or socio-economic background, in order to obtain a more precise emotions map (as shown, for example, in FIG. 3).

In additional embodiments, the predicted emotional and/or physiological responses to test odors(s) and/or test taste(s) can be converted into recommendations about the desirability or attractiveness of a product that produces the odor(s) or taste(s). For example, if the algorithm predicts that test odor x elicits feelings of, for example, happiness, comfort, and/or safety in a test subject; a recommendation is made, to the test subject, to obtain a product that produces odor x. Alternatively, if the algorithm predicts that test odor y elicits feelings of, for example, anger, fear, and/or revulsion in a test subject; a recommendation is made, to the test subject, to avoid obtaining a product that produces test odor y.

The combination of data sets with the presentation of taste, smell, sound, images and/or tactile signal can be used to predict a subject's physiological state (e.g., happiness or sadness). The methods can be used to design a set of optimal stimuli to provide a desired response.

The method can be used to the creation of a precise emotions flower for general emotions (as shown in FIG. 1) and/or for smell/taste related emotions. The method can be used to map between a selected database of compounds and their corresponding emotions. The method can be applied to different group of people, such as based on ethnicities, cultures, socio-economic background, in order to get a more precise emotions map (as shown in FIG. 3).

Models can be iteratively updated to reflect changing preferences of an individual or populations. This can be done by including in the training data set data about emotional responses to sensory stimuli posted to social media sites by subjects, or data from persons sharing a group status as a subject. Periodically, the training dataset can be updated to add or replace existing social media data with newer social media data. For example, the training datasets may be updated at least once over a month, at least once over six months, at least once over a year, at least once over two years or at least once over five years.

### III. Methods of Inferring Emotional Response to a Sensory Stimulus Using a Classifier

A. Inferring Emotional Responses of Individuals

In certain embodiments, the classification algorithms produced by learning algorithms as described herein are used to predict emotional responses to sensory stimuli. For example, to predict an emotional response of a test subject to a test odor, a subject profile, containing trait information, is obtained from the test subject. The algorithm is then provided with (1) the name of the test odor for which a response prediction is sought and (2) the profile containing the trait information of the test subject. Based on these two inputs, the algorithm returns one or more emotional states predicted to be induced by the test odor.

The algorithms can be designed to return the inferred emotional response(s) in a number of different ways. For example, the inferred emotional response can be one of “positive,” “neutral,” or “negative.” Positive emotional responses include, for example, amused, blissful, calm, cheerful, content, dreamy, ecstatic, energetic, excited, flirty, giddy, good, happy, joyful, loving, mellow, optimistic, peaceful, silly, and sympathetic. A neutral emotional response can be indifference. Negative emotional responses include, for example, angry, annoyed, apathetic, bad, cranky, depressed, envious, frustrated, gloomy, grumpy, guilty, indifferent, irritated, melancholy, pessimistic, rejected, restless, sad, stressed, and weird.

Models may predict that certain combinations of stimuli among smells and tastes elicit a positive or negative emotional response, and that within preferred ranges, sub-combinations cluster in attractiveness based, in part on biology and ethnicity or culture. Referring to FIG. 3, taste combinations can range on scales of sweetness and sourness. Combinations that provoke a positive emotional response in individuals may generally fall into a region, referred to here as a “biological optimum”. However, within that optimum, different combinations may be preferred depending on cultural influence. For example, lutefisk is attractive to people sharing Scandinavian culture, but may not be as attractive to persons sharing other cultures.

B. Inferring Emotional Responses of Groups

In a fashion similar to inferring emotional responses of a subject, the classification algorithms can also infer emotional responses of a group, e.g., a consumer group. In these embodiments, trait information of a group is input into the classification algorithm, along with an identifier of one or more sensory stimuli. The algorithm then provides one or more inferred emotional responses (subjective responses and/or objective responses) of the consumer group to the one or more sensory stimuli being tested. That is, for any particular consumer group, the algorithm will, upon input of a particular sensory stimulus, infer the resulting emotional response(s) that will be invoked in the group by said stimulus.

In some embodiments, the group is a consumer group. A consumer group is a target market of individuals that share common traits. Certain of the exemplary individual traits, described above, can also be applied to groups. Characteristics of consumer markets based on demographics include gender, age, ethnic background, income, occupation, education, household size, religion, generation, nationality and social class.

In certain cases, depending on the nature of the group, all members of a consumer group will share a particular trait (for example, if the consumer group is made up of males only, or if all members of the consumer group have the same educational level, or if all members of the consumer group are members of the same religion). In other cases, not all members of a consumer group will share a particular trait. In these cases, two approaches can be used. In the first, a threshold level is set and, if the trait is shared by a percentage of group members that exceeds the threshold, the group is deemed to possess that trait. A threshold is set at a value commensurate with the perceived importance of the trait and can be 50%, 60%, 70%, 75%, 80%, 90%, 95%, 99% or any integral value therebetween. Another approach is to weight the value of the trait in proportion to the percentage of group members that possess that trait. For example, if a consumer group consists entirely of males, but only half of those males possess a particular SNP, the presence of the particular SNP would be given 50% the weight of gender in training the algorithm.

Accordingly, the classification algorithm treats a group as it would an individual having the defining traits of the group. So, for example, where any individual trait is characteristic of a group, this trait can be used in the test vector upon which the classification algorithm operates. If, for example, an ethnic trait, a genetic trait or socioeconomic trait is a predictor of emotional response to a sensory stimulus, then, such a trait predicts the emotional response of persons sharing those characteristic traits.

Group traits include traits shared by all members of the group and traits that are not possessed by all members of the group. Traits shared by all members of the group include, depending on the nature of the group, gender, age, occupation, education, religion, ethnicity, place of residence, nationality, household size and environmental exposure. Traits not possessed by all members of the group include, depending on the make-up of the group, genetic traits, epigenetic traits, proteomic traits, phenotypic traits, cultural traits, socioeconomic level, environmental exposure, geographic area of residence, gender, age, ethnic background, income, occupation, education, household size, religion, generation, nationality and social class. For traits that are not possessed by all members of the group, the influence of the trait can be weighted based on its frequency, or can be required to exceed a threshold before being considered in the analysis.

In certain embodiments, group information comprises genetic information. Genetic information (i.e., genetic traits) includes identification of allelic variants of one or more marker genes, and single nucleotide polymorphisms (SNPs). Group traits also include epigenetic information and phenotypic information. Group information also includes information related to environment or to environmental exposure to a substance such as, for example, automobile exhaust, agricultural chemicals, pesticides and radiation.

As with individuals, inferred emotional responses of a group include, positive, neutral and negative; wherein positive responses include one or more of: amused, blissful, calm, cheerful, content, dreamy, ecstatic, energetic, excited, flirty, giddy, good, happy, joyful, loving, mellow, optimistic, peaceful, silly, and sympathetic; a neutral response is indifference; and negative responses include one or more of: angry, annoyed, apathetic, bad, cranky, depressed, envious, frustrated, gloomy, grumpy, guilty, indifferent, irritated, melancholy, pessimistic, rejected, restless, sad, stressed, and weird.

In certain embodiments, the classification algorithms disclosed herein are applied to a consumer group profile to identify one or more sensory stimuli that are inferred to elicit a positive emotional response from members of the group. With this information, a merchant can stock one or more products that possess the sensory stimulus or stimuli predicted to elicit the positive emotional response. Conversely, the classification algorithms disclosed herein can be applied to a consumer group profile to identify one or more sensory stimuli that are inferred to elicit a negative emotional response from members of the group. With this information, a merchant can avoid stocking, or remove from inventory, products that possess the sensory stimulus or stimuli predicted to elicit the negative emotional response.

### IV. Methods of Customizing Products for Target Individuals or Markets

A. Recommending Products to Individuals or Groups

Having obtained an inferred emotional response, by a subject, to a particular sensory stimulus, the algorithm makes it possible to provide a recommendation to a subject regarding an object (e.g., a product) comprising said sensory stimulus. The recommendation communicates to the subject the type of emotional response she or he is likely to have to the object (e.g., a positive response or a negative response), based on the subject's individual traits.

The classification algorithms disclosed herein can assist a customer in the selection of a product, from among a plurality of products. For example, if a product line comprises a plurality of products, each of which comprises a different sensory stimulus; a potential customer can provide a subject profile containing individual trait information (as described elsewhere herein) and the subject profile, along with the different sensory stimuli associated with each product, are provided to the classification algorithm. The classification algorithm is then executed to predict the customers emotional response to the product. The predicted emotional response can be communicated to the customer. The customer can then order or purchase one or more of those products. Product selection in this fashion can be conducted in person or electronically.

Such a recommendation could be made at a kiosk in a store in which a customer enters trait information which the algorithm can use to predict emotional response. Alternatively, the system could be web-based in which a webpage displays a number of different products having different smells or tastes, receives, through an internet connection, subject's trait information, executes a classifier to predict an emotional response to each different product, and transmits over the web a message to a display accessed by the user to recommend a product. Alternatively, products predicted to produce a positive emotional response or, at least, not a negative emotional response, can be promoted to the customer, for example, by highlighting or directing to particular webpages or with pop-up windows. The recommendation may include the subjects predicted emotional response to the product. For example, products might be indicated as being “energizing” or “calming”.

The classification algorithms disclosed herein are also useful to merchants by providing information on which of the merchant's products (for a product that possesses a sensory stimulus such as a smell or a taste) should be offered to or provided to a customer. For example, a merchant obtains a subject profile from a potential customer, and provides the subject profile, along with information on sensory stimuli associated with various of the merchant's products, to a classification algorithm as disclosed herein; and the algorithm provides an inferred emotional response of the customer to each of the products. Those products which are predicted to provide a positive emotional response are then offered, recommended, or provided to the customer. In certain embodiments, provision of the product may be for promotional purposes; in other embodiments, payment is made by the customer to the merchant, upon provision of the product. The aforementioned process can be conducted on a computer system as described elsewhere herein.

B. Producing Products Customized for Individuals or Groups

The algorithm also allows a merchant to modify a product to make it more appealing to a subject (e.g., potential customer) by adding to the product one or more sensory stimuli that elicit a positive response for said subject, and/or by removing from the product one or more sensory stimuli that elicit a negative response for said subject. In further embodiments, amounts of sensory stimuli in a product can be modulated to affect the emotional response of a subject along one or a plurality of different dimensions.

So, for example, referring to FIG. 2, it may be predicted that a product will elicit in an individual, or group, feelings of “disgust” and “serenity”. Amounts of a compound in the product that elicits “disgust” can be decreased so that an emotional response of “boredom” is predicted. Amounts of a substance predicted to elicit “serenity” can be increased to produce a predicted response of “joy”, or, it can be replaced with another compound predicted to elicit “joy” or “ecstasy”. Furthermore, a compound predicted to elicit a feeling of “trust” can further be added to the product.

Accordingly, a predicted emotional response profile of a product can be customized by altering kinds and/or amounts of compounds predicted to elicit a desired emotional response.

### V. Computer and Web-Based Products

Also provided are computer systems comprising a processor; a memory coupled to the processor, and computer-executable instructions for implementing a classification algorithm on a subject profile or a consumer group profile, as disclosed herein. The memory stores a module comprising a subject profile, which includes data about individual traits of the subject (or a consumer group profile, which includes data about shared, threshold or weighted traits of the consumer group) and a classification rule which, based on the subject profile or the consumer group profile, predicts an emotional response by the subject, or by the consumer group, to a sensory stimulus. Optionally, the computer system comprises a display or other means for conveying and/or transmitting information.

Also provided are computer-readable media comprising machine-executable code that, upon execution by a computer processor, implements a classification rule generated by a method as described herein to predict emotional response to a sensory stimulus. In certain embodiments, the media are in tangible, non-transitory form.

Processors and computer systems: The present disclosure provides computer control systems that are programmed to implement methods of the disclosure. The computer system can regulate various aspects of data collection, data analysis, and data storage, of subject profiles, sensory stimulus data and emotional responses. The computer system can be an electronic device of a user, or a computer system that is remotely located with respect to the electronic device. The electronic device can be a mobile electronic device.

In some embodiments, the hardware and software code of the computer system is built around a field-programmable gate array (FPGA) architecture. Unlike microprocessors, which process a fixed set of instructions using a corresponding hard-wired block of logic gates, an FPGA does not have any hard-wired logic blocks. Rather, the logic blocks are programmed by the user, which constitutes the “programming” of an FPGA (the code is essentially a hardware change). FPGAs have the advantage of being much faster than microprocessors for performing specific sets of instructions.

In some embodiments, the computer system comprises a central processing unit (CPU). FIG. 7 shows a computer system that can include a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., portable PC, tablet PC, smart phone). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor)-executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240. Examples of UIs include, without limitation, a graphical user interface (GUI) and web-based user interface.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The phrase “at least one” includes “one or more” and “one or a plurality”. The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Headings used herein are for organizational purposes only and are not meant to be used to limit the scope of the description.

We claim:

1. A method for inferring an emotional response of a subject to a sensory stimulus comprising:
a) for each of a set of subjects in a cohort of subjects:
(i) exposing the subject to one or more sensory stimuli;
(ii) eliciting and electronically recording subjective response data from the subject to each sensory stimulus and receiving the recorded subjective response data into computer memory;
(iii) electronically measuring objective response data from the subject to each sensory stimulus and receiving the measured objective response data into computer memory, wherein subjective responses and objective responses indicate an emotional response to the sensory stimulus;
(iv) receiving into computer memory responses including trait data about the subject; and
(v) receiving into computer memory data about each sensory stimulus; wherein the received data for a subject constitutes a subject dataset;
b) generating a training dataset by collecting the subject datasets;
c) training a machine learning algorithm on the training dataset to produce a model that infers an emotional response of a subject based on one or more individual trait data;
d) at a user interface associated with a target subject (e.g., who is not in the cohort), inferring an emotional response by the subject to a sensory stimulus based on individual trait data provided by the target subject.

2. A method comprising:
a) determining a profile comprising trait information about a plurality of individual traits for each of one or more subjects or consumer groups; and
b) for each of one or more sensory stimuli, wherein each stimulus is an odor or a taste, predicting emotional response by each of the subjects or consumer groups to each of the sensory stimuli, based on the trait information.

3. The method of claim 2, further comprising:
c) translating the predicted emotional responses into recommendations to each subject or consumer group about attractiveness of products incorporating the sensory stimuli.

4. A method of generating an emotional response prediction model comprising:
a) providing a dataset that comprises, for each of a plurality of subjects, data including:
(i) a subject profile comprising data on a plurality of individual traits from the subject;
(ii) sensory stimulus data for each of one or a plurality of sensory stimuli to which the subject is exposed; and
(iii) emotional response data for each subject indicating emotional response by the subject to each of the sensory stimuli to which the subject is exposed, wherein the emotional response data comprises one or both of subjective response data and objective response data; and
b) training a learning algorithm to generate a model that infers a subject's emotional response to a sensory stimulus based on the subject's profile.

5. The method of claim 4, wherein the sensory stimulus in an odor.

6. The method of claim 4, wherein the sensory stimulus is a taste.

7. The method of claim 4, wherein the emotional response comprises a subjective response comprising a linguistic expression selected from spoken, written, or signed.

8. The method of claim 4, wherein the emotional response data comprises one or a plurality of objective responses selected from the group comprising facial expressions, micro expressions, brain signals, electroencephalography (EEG) signals, functional magnetic resonance imaging (fMRI) signals, body chemical stimuli, body chemical production, pupil dilation, skin conductance, skin potential, skin resistance, skin temperature, respiratory frequency, blood pressure, blood flow, saliva production and flow rate, and any combination thereof.

9. The method of claim 4, wherein the emotional response comprises data derived from social media activity of the subject or a group to which the subject belongs.

10. The method of claim 4, wherein the emotional response is classified into a discrete or continuous range.

11. The method of claim 9, wherein the emotional response is classified as a number, a degree, a level, a range or a bucket.

12. The method of claim 9, wherein the emotional response is classified as an image selected by the subject from a group of images.

13. The method of claim 9, wherein the emotional response is classified as a subjective feeling verbalized by the subject.

14. The method of claim 4, wherein the emotional response is classified into a category within a set of discrete categories, wherein the discrete categories are hierarchically arranged from least positive to most positive emotional response.

15. The method of claim 14, wherein the set comprises any of 3, 4, 5, 6, 7, 8, 9 or 10 discrete categories.

16. The method of claim 14, wherein the set comprises two discrete categories, including a negative emotional response and a positive emotional response.

17. The method of claim 14, wherein the set comprises three discrete categories, including a negative emotional response, a neutral emotional response and a positive emotional response.

18. The method of claim 4, wherein the emotional response is classified into a multi variate response, with each variable being measured on a range.

19. The method of claim 18, wherein the variables include one or a plurality of responses selected from love, submission, awe, disapproval, remorse, contempt, aggressiveness, and optimism.

20. The method of claim 4, wherein the emotional response is selected from one or more of: amused, blissful, calm, cheerful, content, dreamy, ecstatic, energetic, excited, flirty, giddy, good, happy, joyful, loving, mellow, optimistic, peaceful, silly, and sympathetic.

21-80. (canceled)
